(19) United States
(12) Patent Application Publication
(10) Pub. No.: US 2015/0342856 A1
(43) Pub. Date: Dec. 3, 2015

(54) Cationic Comb Copolymers, Preparation Thereof And Use Thereof In Cosmetic, Pharmaceutical And Dermatological Formulations

(71) Applicants: Peter KLUG, Grossostheim (DE); Thomas LINDNER, Wiesbaden (DE); Matthias LOEFFLER, Idstein (DE); Dirk FISCHER, Klein-Winternheim (DE); Matthias KUNZE, Frankfurt am Main (DE); Carina MILDNER, Frankfurt am Main (DE); Sebastiano LO VASCO, Glauburg (DE); Tomas CERNY, Frankfurt am Main (DE)

(72) Inventors: Peter KLUG, Grossostheim (DE); Thomas LINDNER, Wiesbaden (DE); Matthias LOEFFLER, Idstein (DE); Dirk FISCHER, Klein-Winternheim (DE); Matthias KUNZE, Frankfurt am Main (DE); Carina MILDNER, Frankfurt am Main (DE); Sebastiano LO VASCO, Glauburg (DE); Tomas CERNY, Frankfurt am Main (DE)

(73) Assignee: CLARIANT FINANCE (BVI) LIMITED, Tortola (VG)

(21) Appl. No.: 14/820,317

(22) Filed: Aug. 6, 2015

Related U.S. Application Data
(62) Division of application No. 13/262,382, filed on Jan. 3, 2012.

(30) Foreign Application Priority Data
Apr. 1, 2009 (DE) 10 2009 015 868.5

(51) Int. Cl. A61K 8/81; A61Q 90/00

(52) U.S. Cl. CPC A61K 8/81; A61Q 90/00; A61K 2800/10

(57) ABSTRACT

Copolymers are described, containing a) 20.0-99.9% by weight of one or more structural units resulting from polymerizable substances of the following structural formula (I) in which R² is a linear or branched saturated alkyl radical having 6 to 200 carbon atoms or a linear or branched, mono- or polyunsaturated alkenyl radical having 6 to 200 carbon atoms, and R¹, Y, n and m are each as defined in claims 1, b) 0.1-20.0% by weight of one or more structural units resulting from polymerizable quaternary ammonium compounds, and c) 0-60.0% by weight of one or more nonionic structural units resulting from one or more further polymerizable substances. The copolymers are advantageously suitable, for example, for the production of cosmetic, dermatological or pharmaceutical formulations.

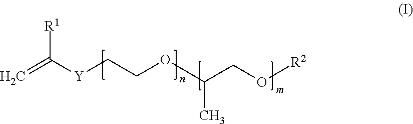

The invention which follows relates to cationic comb copolymers, to a process for preparing such cationic comb copolymers, and to the use of these copolymers in cosmetic, pharmaceutical, and dermatological formulations.

Consumer desires and rheology of cosmetic products are closely interlinked. Thus, for example, the visual appearance of a shampoo or of a cream wash and the handling qualities of an oil are influenced by the viscosity. The sensorial properties, such as consistency or spreadability, determine the individual profile of a cosmetic product. Not only the effectiveness of active substances (e.g., sun protection filters) but also the storage stability of the formulation are closely correlated with the rheological properties of the products.

In the cosmetics segment, cationic polymers are accorded a key role as conditioners. State of the art are, in particular, conditioners based on polydiallyldimethylammonium chloride and the water-soluble copolymers thereof. The diversity of possible structures and the diverse possibilities for application that are associated with these structures are manifested not least in a multiplicity of patents filed worldwide since the mid-1970s.

A substantial drawback of the conditioners based on polydiallyldimethylammonium chloride is that, in oil-based formulations, they exhibit an inadequate thickening capacity. In order to improve the consistency of the formulations, fats and waxes are often added, which in turn bring with them the drawback of a dull and sticky skin feel.

The object was to provide substances for cosmetic, pharmaceutical, and dermatological formulations that exhibit conditioning properties for the skin and for the hair and that also have good consistency-imparting properties even in oil-based formulations or in formulations with a high oil fraction.

It has surprisingly been found that this object is achieved by certain cationic comb copolymers comprising structural units which derive, formally, from polymerizable quaternary ammonium compounds and from polymerizable nonionic compounds which comprise hydrocarbon groups having at least six carbon atoms.

The invention provides copolymers comprising

a) 20.0%-99.9% by weight of one or more structural units originating from polymerizable substances of the following structural formula (I)

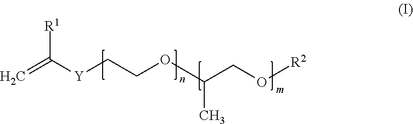

in which
R¹ is hydrogen or methyl,
Y is a chemical bond, O, NR³, S, PR³, CH₂, CH₂O, CH₂NR³, CH₂S, C(O), C(NR³), C(O)O, C(O)NR³, C(NR⁴)NR³, C(O)S, R⁶OC(O)O, R⁶OC(O)NR³, R⁶R³NC(O)NR⁴, R⁶NR³C(NR⁵)NR⁴, R⁶OC(O)S, R⁶P(O)O, R⁶OP(O)O, R⁶S(O), R⁶S(O)(O), R⁶S(O)O, R⁶S(O)(O)O, R⁶OS(O)O or R⁶OS(O)(O)O,
R² is a linear or branched saturated alkyl radical, having 6 to 200 carbon atoms, or a linear or branched mono- or polyunsaturated alkenyl radical having 6 to 200 carbon atoms,
R³, R⁴ and R⁵ each independently of one another are hydrogen or a linear or branched saturated alkyl radical having 1 to 200 carbon atoms, or a linear or branched mono- or polyunsaturated alkenyl radical having 2 to 200 carbon atoms,
R⁶ is a chemical bond or CH₂, and
n and m each independently of one another are an integer from 0 to 200,
   b) 0.1%-20.0% by weight of one or more structural units, originating from polymerizable quaternary ammonium compounds, and
   c) 0%-60.0% by weight of one or more nonionic structural units originating from one or more further polymerizable substances.

The term “originating from one or more further polymerizable substances” set out above in component c) means, for the purposes of the present invention, that component c) does not comprise any compound which takes on a definition of the above-stated formula (I).

The copolymers of the invention are colorless to opaque and transparent, produce a good skin feel, and give the hair shine and good combability. They are suitable for preparing a very wide variety of cosmetic, pharmaceutical, and dermatological formulations, and are highly compatible in particular with water/oil-based or oil-based formulations. They display an outstanding oil-binding capacity and are able to thicken oils effectively. Furthermore, they display a good dispersing capacity and are suitable for imparting consistency stably to emulsions and creams. They are further suitable for the formulation of water-resistant sun protection products.

WO 2004/041220 and WO 2004/041150 describe cosmetic preparations, more particularly lipsticks and makeup preparations, which in addition to a liquid fatty phase comprise a semicrystalline polymer of a C₁₄-C₂₄-α-olefin and of a further monomer selected from carboxylic esters, preferably C₁₄-C₂₄ alkyl- or C₁₁-C₁₅ perfluoroalkyl (meth)acrylates, and N-alkyl(meth)acrylamides.

EP 1 681 046 describes cosmetic, pharmaceutical, and dermatological preparations which comprise copolymer waxes. The copolymer waxes comprise structural units derived formally from α-olefins having 26 to 60 carbon atoms, derivatives of (meth)acrylic acid such as esters, amides or salts, and optionally further monomers.

EP 1 693 047 describes cosmetic, pharmaceutical, and dermatological preparations which comprise copolymer waxes. The copolymer waxes comprise structural units derived from α-olefins having 26 to 60 carbon atoms, from maleic anhydride, maleic acid or salts thereof, and optionally from further monomers.

Preferred copolymers of the invention comprise 20.0% to 99.9% by weight of one or more structural units of component a) originating from polymerizable substances of the following structural formula (I)

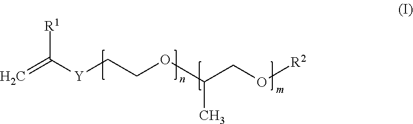

in which
R¹ is hydrogen or methyl,
Y is a chemical bond, O, NR³, S, CH₂O, CH₂NR³, CH₂S, C(O), C(NR³), C(O)O or C(O)NR³, preferably a chemical bond, O, NR³, CH₂O, CH₂NR³, C(O)O or C(O)NR³, and more preferably a chemical bond C(O)O or C(O)NR³,
R² is a linear or branched saturated alkyl radical having 6 to 200, preferably having 8 to 58, more preferably having 12 to 40, and with particular preference having 12 to 30 carbon atoms, or a linear or branched mono- or polyunsaturated alkenyl radical having 6 to 200, preferably having 8 to 58, more preferably having 12 to 40, and with particular preference having 12 to 30 carbon atoms,
R³ is hydrogen or methyl, and
n and m each independently of one another are an integer from 0 to 200, preferably from 0 to 100, more preferably from 0 to 50, and with particular preference from 0 to 30.

In the compounds of the formula (I), Y with particular preference is a chemical bond or C(O)O.

Particularly preferred copolymers of the invention comprise 20.0% to 99.9% by weight of one or more structural units of component a) originating from one or more polymerizable compounds selected from the structural formulae (Ia), (Ib), and (Ic)

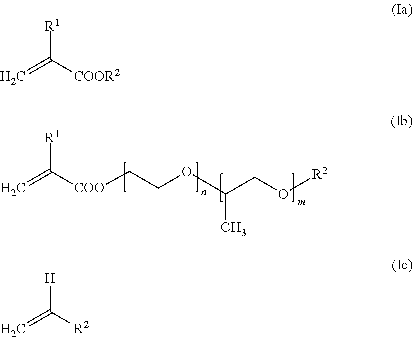

in which
R¹ is hydrogen or methyl, and
R² is a linear or branched saturated alkyl radical having 6 to 200, preferably having 8 to 58, more preferably having 12 to 40, and with particular preference having 12 to 30 carbon atoms, or a linear or branched mono- or polyunsaturated alkenyl radical having 6 to 200, preferably having 8 to 58, more preferably having 12 to 40, and with particular preference having 12 to 30 carbon atoms, and
n and m each independently of one another are an integer from 0 to 200, preferably from 0 to 100, more preferably from 0 to 50, and with particular preference from 0 to 30, and the sum n+m is a number from 1 to 200, preferably from 2 to 100, more preferably from 3 to 50, and with particular preference from 3 to 30.

In one preferred embodiment of the invention, preference among the compounds of the formula (Ia) is given to the (meth)acrylic esters of the alkyl alcohols, i.e., R² in these preferred compounds of the formula (Ia) is an alkyl radical. In one particularly preferred embodiment of the invention, R² in these compounds is a linear alkyl radical.

The prefix “(meth)acryl” for the purposes of the present invention is an abbreviational designation for the two prefixes, “acryl” and “methacryl”.

In a further preferred embodiment of the invention, among the compounds of the formula (Ia), the (meth)acrylic esters of lauryl, myristyl, cetyl, stearyl, isostearyl, oleyl, octyldodecyl, behenyl, and myricyl alcohols are preferred.

In a further preferred embodiment of the invention, among the compounds of the formula (Ia), the (meth)acrylic esters of alcohols having 12 to 22 carbon atoms are preferred. In this preferred embodiment of the invention, among the compounds of the formula (Ia), the (meth)acrylic esters of lauryl, myristyl, cetyl, stearyl, isostearyl, oleyl, octyldodecyl, and behenyl alcohols are particularly preferred, and the (meth)acrylic esters of lauryl, cetyl, stearyl, isostearyl, octyldodecyl, and behenyl alcohols are especially preferred. Of the (meth)acrylic esters just stated, preference is given in turn to those of the linear alkyl alcohols.

In another preferred embodiment of the invention, among the compounds of the formula (Ib), the substances selected from (meth)acrylic esters of alkyl-polyethylene glycols, of alkyl-polypropylene glycols and of alkyl-polyethylene-polypropylene glycols are preferred, i.e., R² in these preferred compounds of the formula (Ib) is an alkyl radical. In one particularly preferred embodiment of the invention, R² in these compounds is a linear alkyl radical.

In a further preferred embodiment of the invention, “m” in the compounds of the formula (Ib) is 0, i.e., these compounds are purely ethoxylated compounds. Where m in the compounds of the formula (Ib) is 0, n is an integer from 1 to 200, preferably from 2 to 100, more preferably from 3 to 50 and with particular preference from 3 to 30.

In another preferred embodiment of the invention, among the compounds of the formula (Ib), the acrylic and/or methacrylic esters of alcohol polyglycol ethers are selected from

a) lauryl ethoxylates, more particularly
Genapol® LA 020 lauryl ethoxylate with 2 EO C_{12/14} alcohol, linear
Genapol® LA 030 lauryl ethoxylate with 3 EO C_{12/14} alcohol, linear
Genapol® LA 040 lauryl ethoxylate with 4 EO C_{12/14} alcohol, linear
Genapol® LA 050 lauryl ethoxylate with 5 EO C_{12/14} alcohol, linear
Genapol® LA 070 lauryl ethoxylate with 7 EO C_{12/14} alcohol, linear
Genapol® LA 080 lauryl ethoxylate with 8 EO C_{12/14} alcohol, linear
Genapol® LA 090 lauryl ethoxylate with 9 EO C_{12/14} alcohol, linear
Genapol® LA 110 lauryl ethoxylate with 11 EO C_{12/14} alcohol, linear
Genapol® LA 200 lauryl ethoxylate with 20 EO C_{12/14} alcohol, linear
Genapol® LA 2310 lauryl ethoxylate with 23 EO C_{12/14} alcohol, linear
Genapol® LA 250 lauryl ethoxylate with 25 EO C_{12/14} alcohol, linear
   b) cocoalkyl ethoxylates, more particularly
Genapol® C 050 C_{12/14/16} alkyl ethoxylate with 5 EO C_{12/14/16} alcohol, linear
Genapol® C 100 C_{12/14/16} alkyl ethoxylate with 10 EO C_{12/14/16} alcohol, linear
Genapol® C 200 C_{12/14/16} alkyl ethoxylate with 20 EO C_{12/14/16} alcohol, linear
Genapol® C 250 C_{12/14/16} alkyl ethoxylate with 25 EO C_{12/14/16} alcohol, linear
Genapol® U 100 C_{14/16/18} alkyl ethoxylate with 10 EO C_{14/16/18} alcohol, unsaturated
Genapol® U 200 C_{14/16/18} alkyl ethoxylate with 20 EO C_{14/16/18} alcohol, unsaturated
Genapol® GS 080 C₁₂₋₂₀ alkyl ethoxylate with 8 EO C₁₂₋₂₀ alcohol, linear
   c) oleyl ethoxylates, more particularly
Genapol® O 020 oleyl ethoxylate with 2 EO C_{16/18} alcohol, linear, unsaturated
Genapol® O 050 oleyl ethoxylate with 5 EO C_{16/18} alcohol, linear, unsaturated
Genapol® O 080 oleyl ethoxylate with 8 EO C_{16/18} alcohol, linear, unsaturated
Genapol® O 100 oleyl ethoxylate with 10 EO C_{16/18} alcohol, linear, unsaturated
Genapol® O 120 oleyl ethoxylate with 12 EO C_{16/18} alcohol, linear, unsaturated
Genapol® O 150 oleyl ethoxylate with 15 EO C_{16/18} alcohol, linear, unsaturated
Genapol® O 200 oleyl ethoxylate with 20 EO C_{16/18} alcohol, linear, unsaturated
Genapol® O 230 oleyl ethoxylate with 23 EO C_{16/18} alcohol, linear, unsaturated
Genapol® O 250 oleyl ethoxylate with 25 EO C_{16/18} alcohol, linear, unsaturated
Genapol® O 300 oleyl ethoxylate with 30 EO C_{16/18} alcohol, linear, unsaturated
   d) tallowalkyl ethoxylates, more particularly
Genapol® T 080 tallowalkyl ethoxylate with 8 EO C_{16/18} alcohol, linear, unsaturated
Genapol® T 110 tallowalkyl ethoxylate with 11 EO C_{16/18} alcohol, linear, unsaturated
Genapol® T 150 tallowalkyl ethoxylate with 15 EO C_{16/18} alcohol, linear, unsaturated
Genapol® T 200 tallowalkyl ethoxylate with 20 EO C_{16/18} alcohol, linear, unsaturated
Genapol® T 250 tallowalkyl ethoxylate with 25 EO C_{16/18} alcohol, linear, unsaturated
Genapol® T 500 tallowalkyl ethoxylate with 50 EO C_{16/18} alcohol, linear, unsaturated
Genapol® T 800 tallowalkyl ethoxylate with 80 EO C_{16/18} alcohol, linear, unsaturated
e) stearyl alcohol ethoxylates, more particularly
Genapol® HS 020 stearyl alcohol with 2 EO C_{16/18} alcohol, linear, saturated
Genapol® HS 200 stearyl alcohol with 20 EO C_{16/18} alcohol, linear, saturated
f) C₁₁ alkyl ethoxylates, more particularly
Emulsogen® EPN 118 C₁₁ alkyl ethoxylate with 11 EO C₁₁ alcohol, linear, little branching
Emulsogen® EPN 287 C₁₁ alkyl ethoxylate with 28 EO C₁₁ alcohol, linear, little branching
Emulsogen® EPN 407 C₁₁ alkyl ethoxylate with 40 EO C₁₁ alcohol, linear, little branching
Emulsogen® LCN 050 C₁₁ alkyl ethoxylate with 5 EO C₁₁ alcohol, branched
Emulsogen® LCN 070 C₁₁ alkyl ethoxylate with 7 EO C₁₁ alcohol, branched
Emulsogen® LCN 088 C₁₁ alkyl ethoxylate with 8 EO C₁₁ alcohol, branched
Emulsogen® LCN 118 C₁₁ alkyl ethoxylate with 11 EO C₁₁ alcohol, branched
Emulsogen® LCN 158 C₁₁ alkyl ethoxylate with 15 EO C₁₁ alcohol, branched
Emulsogen® LCN 217 C₁₁ alkyl ethoxylate with 21 EO C₁₁ alcohol, branched
Emulsogen® LCN 287 C₁₁ alkyl ethoxylate with 28 EO C₁₁ alcohol, branched
Emulsogen® LCN 407 C₁₁ alkyl ethoxylate with 40 EO C₁₁ alcohol, branched
Emulsogen® LCN 506 C₁₁ alkyl ethoxylate with 50 EO C₁₁ alcohol, branched
Genapol® UD 030 C₁₁ alkyl ethoxylate with 3 EO C₁₁ alcohol, linear, little branching
Genapol® UD 050 C₁₁ alkyl ethoxylate with 5 EO C₁₁ alcohol, linear, little branching
Genapol® UD 079 C₁₁ alkyl ethoxylate with 7 EO C₁₁ alcohol, linear, little branching
Genapol® UD 080 C₁₁ alkyl ethoxylate with 8 EO C₁₁ alcohol, linear, little branching
Genapol® UD 088 C₁₁ alkyl ethoxylate with 8 EO C₁₁ alcohol, linear, little branching
Genapol® UD 110 C₁₁ alkyl ethoxylate with 11 EO C₁₁ alcohol, linear, little branching
g) isotridecyl ethoxylates, more particularly
Genapol® X 020 isotridecyl ethoxylate with 2 EO C₁₃ alcohol, branched
Genapol® X 050 isotridecyl ethoxylate with 5 EO C₁₃ alcohol, branched
Genapol® X 060 isotridecyl ethoxylate with 6 EO C₁₃ alcohol, branched
Genapol® X 065 isotridecyl ethoxylate with 6.5 EO C₁₃ alcohol, branched
Genapol® X 080 isotridecyl ethoxylate with 8 EO C₁₃ alcohol, branched
Genapol® X 089 isotridecyl ethoxylate with 8 EO C₁₃ alcohol, branched
Genapol® X 090 isotridecyl ethoxylate with 9 EO C₁₃ alcohol, branched
Genapol® X 100 isotridecyl ethoxylate with 10 EO C₁₃ alcohol, branched
Genapol® X 150 isotridecyl ethoxylate with 15 EO C₁₃ alcohol, branched
Genapol® X 158 isotridecyl ethoxylate with 15 EO C₁₃ alcohol, branched
Genapol® X 307 isotridecyl ethoxylate with 30 EO C₁₃ alcohol, branched
Genapol® X 407 isotridecyl ethoxylate with 40 EO C₁₃ alcohol, branched
Genapol® X 1003 isotridecyl ethoxylate with 100 EO C₁₃ alcohol, branched
Genapol® X 1005 isotridecyl ethoxylate with 100 EO C₁₃ alcohol, branched
Genapol® 1879 isotridecyl ethoxylate with 15 EO C₁₃ alcohol, branched
Genapol® 3214 isotridecyl ethoxylate with 25 EO C₁₃ alcohol, branched
   h) isodecyl ethoxylates, more particularly
Genapol® ID 030 isodecyl ethoxylate with 3 EO C₁₀ alcohol, branched
Genapol® ID 060 isodecyl ethoxylate with 6 EO C₁₀ alcohol, branched
Genapol® ID 070 isodecyl ethoxylate with 7 EO C₁₀ alcohol, branched
Genapol® EP 1022 isodecyl ethoxylate with 2 EO, 2PO C₁₀ alcohol, branched
   i) oxo-process alcohol ethoxylates, more particularly
Genapol® OX 030 C_{12/15} alkyl ethoxylate with 3 EO C_{12/15} alcohol, little branching
Genapol® OX 050 C_{12/15} alkyl ethoxylate with 5 EO C_{12/15} alcohol, little branching
Genapol® OX 070 C_{12/15} alkyl ethoxylate with 7 EO C_{12/15} alcohol, little branching
Genapol® OX 080 C_{12/15} alkyl ethoxylate with 8 EO C_{12/15} alcohol, little branching
Genapol® OX 100 C_{12/15} alkyl ethoxylate with 10 EO C_{12/15} alcohol, little branching
Genapol® OX 109 C_{12/15} alkyl ethoxylate with 10 EO C_{12/15} alcohol, little branching
Genapol® OA 030 C_{14/15} oxo-process alcohol with 3 EO C_{14/15} oxo-process alcohol
Genapol® OA 3070 C_{14/15} oxo alcohol, 3EO, 7PO C_{14/15} oxo-process alcohol
Genapol® OA 040 C_{14/15} oxo-process alcohol with 4 EO C_{14/15} oxo-process alcohol
Genapol® OA 050 C_{14/15} oxo-process alcohol with 5 EO C_{14/15} oxo-process alcohol
Genapol® OA 070 C_{14/15} oxo-process alcohol with 7 EO C_{14/15} oxo-process alcohol
Genapol® OA 080 C_{14/15} oxo-process alcohol with 8 EO C_{14/15} oxo-process alcohol
Hostacerin® T3 cetyl alcohol with 3 EO C₁₆ alcohol, linear, saturated
Hostacerin® CS 200 cetyl alcohol with 20 EO C₁₆ alcohol, linear, saturated
Emulsogen® HCO 040 PEG-40 castor oil, hydrogenated C_{16/18} hydroxyalcohol, linear, saturated
Emulsogen® HCO 060 PEG-60 castor oil, hydrogenated C_{16/18} hydroxyalcohol, linear, saturated and
   j) alcohol alkoxylates, EO/PO, more particularly
Genapol® EO 0244 C_{10/12} alcohol with 4 EO and 4 PO C_{10/12} alcohol
Genapol® EP 1182 C₁₁ oxo-process alcohol with 8 EO and 2 PO C₁₁ oxo-process alcohol
Genapol® EP 2464 C_{12/14} alcohol having 6 EO and 4 PO C_{12/14} alcohol
Genapol® EP 2544 C_{12/15} oxo-process alcohol with 4 EO and 4 PO C_{12/15} oxo-process alcohol
Genapol® EP 2564 C_{12/15} oxo-process alcohol with 6 EO and 4 PO C_{12/15} oxo-process alcohol
Genapol® EP 2584 C_{12/15} oxo-process alcohol with 8 EO and 4 PO C_{12/15} oxo-process alcohol
Genapol® EP2525 C_{12/15} oxo-process alcohol with 2 EO and 5 PO C_{12/15} oxo-process alcohol
Genapol® EP 2552 C_{12/15} oxo-process alcohol with 5 EO and 2 PO C_{12/15} oxo-process alcohol
   and also
   (C₁₀-C₁₈) fatty alcohol polyglycol ethers having 8 EO units,
   iso-(C₁₆-C₁₈) fatty alcohol polyglycol ethers having 25 EO units,
   (C₁₈-C₂₂) fatty alcohol polyglycol ethers having 25 EO units,
   octyl alcohol ethers having 3 EO units,
   octyl alcohol ethers having 10 EO units and 3 PO units,
   behenyl alcohol ethers having 10 EO units,
   behenyl alcohol ether having 20 EO units, and
   behenyl alcohol ether having 25 EO units
   are preferred.

In the list above (subgroups a) to j)), relating to preferred acrylic and/or methacrylic esters of alcohol polyglycol ethers and of alkoxylated alcohols in accordance with formula (Ib), the trade names of the alkoxylated alcohols are given on the left, the chemical identification of the alkoxylated alcohols in the middle, and a definition of the alcohols on the right. The products represent commercial products from Clariant.

In the context of the present invention, the abbreviation “EO” denotes ethylene oxide C₂H₄O—, and “PO” propylene oxide C₃H₆O—.

In one particularly preferred embodiment of the invention, of the compounds of the formula (Ib), preference is given to those selected from lauryl methacrylate ethoxylated with 7 EO units; talyl methacrylate ethoxylated with 8 EO units; talyl methacrylate ethoxylated with 25 EO units; and behenyl methacrylate ethoxylated with 25 EO units.

For the purposes of the present invention, the designation “talyl” is used synonymously for the designation “tallowalkyl”.

In a further preferred embodiment of the invention, among the compounds of the formula (Ic), preference is given to those in which R² in an alkyl radical. These compounds are α-olefins. In one particularly preferred embodiment of the invention, R² in these compounds is a linear alkyl radical.

In a further particularly preferred embodiment of the invention, among the compounds of the formula (Ic), preference is given to those selected from 1-hexene, 1-octene, 1-decene, 1-dodecene, 1-tetradecene, 1-hexadecene, 1-octadecene, C₂₀ α-olefin, C₂₂ α-olefin, C₂₄ α-olefin, C₂₆ α-olefin, C₂₈ α-olefin, C₃₀ α-olefin, and also mixtures of α-olefines, including preferably C₁₄₋₁₆ α-olefins, C₂₀₋₂₄ α-olefins, C₂₄₋₂₈ α-olefins, C₂₆₋₂₈ α-olefins, and isomerized α-olefins, including preferably 4-methyl-1-pentene, isomerized C₁₆ α-olefins, isomerized C₁₈ α-olefins, and isomerized C₁₆₋₁₈ α-olefins, such as, for example, isomerized C₁₆₋₁₈ α-olefins in a C₁₆:C₁₈ weight ratio of 65:35.

In another particularly preferred embodiment of the invention, of the compounds of the formula (Ic), preference is given to those selected from α-olefins having 10 to 60 carbon atoms, i.e., R² in these compounds of the formula (Ic) is a saturated alkyl radical having 8 to 58 carbon atoms, and the structural units originating from these compounds of the formula (Ic) are those of the formula —CH₂CHR²—, in which R² is a saturated alkyl radical having 8 to 58 carbon atoms.

In one especially preferred embodiment of the invention among the compounds of the formula (Ic), preference is given to those in which R² is an alkyl radical having 12 to 40 carbon atoms. In an embodiment of the invention which is preferred among these in turn, from among the compounds of the formula (Ic), preference in turn is given to those in which R² is an alkyl radical having 12 to 30 carbon atoms.

In an embodiment of the invention which is preferred in turn among these, from among the compounds of the formula (Ic), preference in turn is given to those in which R² is an alkyl radical having 24 or 26 carbon atoms or stands for mixtures of alkyl radicals having 24 and 26 carbon atoms. In a further embodiment of the invention that is preferred in turn among these, from among the compounds of the formula (Ic), preference is given in turn to those in which R² is an alkyl radical having 28 carbon atoms.

In an extraordinarily preferred embodiment of the invention, from among the compounds of the formula (Ic), preference is given to those selected from C₂₆ α-olefin, C₂₈ α-olefin, C₂₆-C₂₈ α-olefin, and C₃₀ α-olefin.

Extraordinarily preferred are the compounds of the formula (Ia) selected from hexyl acrylate, hexyl methacrylate, decyl acrylate, decyl methacrylate, lauryl acrylate, lauryl methacrylate, hexadecyl acrylate, hexadecyl methacrylate, stearyl acrylate, stearyl methacrylate, isostearyl acrylate, isostearyl methacrylate, octyldodecyl acrylate, octyldodecyl methacrylate, behenyl acrylate and behenyl methacrylate,

the compounds of the formula (Ib) selected from acrylic ester of lauryl alcohol with 7 EO units, methacrylic ester of lauryl alcohol with 7 EO units, acrylic ester of talyl alcohol with 8 EO units, methacrylic ester of talyl alcohol with 8 EO units, acrylic ester of talyl alcohol with 25 EO units, methacrylic ester of talyl alcohol with 25 EO units, acrylic ester of behenyl alcohol with 25 EO units and methacrylic ester of behenyl alcohol with 25 EO units, and
the compounds of the formula (Ic) selected from C₂₆ α-olefin, C₂₈ α-olefin, C₂₆-C₂₈ α-olefin, and C₃₀ α-olefin.

Preferred among these in turn are the compounds of the formula (Ia) selected from lauryl acrylate, lauryl methacrylate, hexadecyl acrylate, stearyl acrylate, isostearyl acrylate, octyldodecyl acrylate, and behenyl acrylate,

the compounds of the formula (Ib) selected from methacrylic ester of lauryl alcohol with 7 EO units, methacrylic ester of talyl alcohol with 8 EO units, methacrylic ester of talyl alcohol with 25 EO units, and methacrylic ester of behenyl alcohol with 25 EO units, and
the compounds of the formula (Ic) selected from C₂₆ α-olefin, C₂₈ α-olefin, C₂₆-C₂₈ α-olefin, and C₃₀ α-olefin.

In a further preferred embodiment of the invention, R² in the compounds of the formula (I) or in the formulae (Ia), (Ib), and (Ic) is a linear or branched saturated alkyl radical having 6 to 200, preferably having 8 to 58, more preferably having 12 to 40, and with particular preference having 12 to 30 carbon atoms. In a particularly preferred embodiment of the invention, R² in the compounds of the formulae (I), (Ia), (Ib), and (Ic) is a linear saturated alkyl radical having 6 to 200, preferably having 8 to 58, more preferably having 12 to 40, and with particular preference having 12 to 30 carbon atoms.

The structural units of component b) preferably originate from substances selected from

b1) polymerizable quaternary ammonium compounds of the following structural formula (II)

X⁻+NR⁷R⁸R⁹R¹⁰ (II)

in which
X⁻ is a negatively charged counterion,
R⁷ is vinyl radical, allyl radical or a radical of the following structural formula (III)

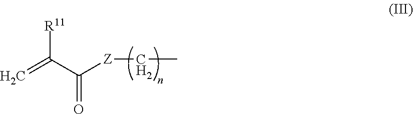

in which
R¹¹ is hydrogen or methyl,
Z is O, NH, NCH₃ or S,
n is an integer from 1 to 50,
R⁸ is a linear or branched alkyl radical having 1 to 50 carbon atoms or is a radical R⁷,
R⁹ is a linear or branched alkyl radical having 1 to 50 carbon atoms or is a radical R⁷, and
R¹⁰ is a linear or branched alkyl radical having 1 to 50 carbon atoms,
   and
   b2) polymerizable cyclic quaternary ammonium compounds with the following structural formula (IV)

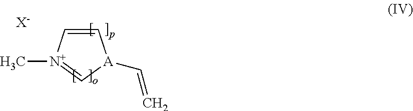

in which
o and p each independently of one another are an integer from 0 to 5,

### A is C, CH, N or P, and

X⁻ is a negatively charged counterion.

In the compounds of the formula (IV), where o is zero or even-numbered, A is C and p is odd-numbered.

In the compounds of the formula (IV), where o is odd-numbered, p may be zero, even-numbered or odd-numbered. If p is zero or even-numbered, A is C. If, however, p is odd-numbered, A may take on the definition CH, N or P.

In the compounds of the formula (IV), where p is zero or is even-numbered, A is C and o is odd-numbered.

In the compounds of the formula (IV), where p is odd-numbered, o may be zero, even-numbered or odd-numbered. If o is zero or even-numbered, A is C. If, however, o is odd-numbered, A may take on the definition CH, N or P.

Preferred negatively charged counterions X⁻in formula (II) and (IV) are chloride, bromide, iodide, ½ SO₄²⁻, and methosulfate.

In one particularly preferred embodiment of the invention, the structural units of component b) originate from polymerizable quaternary ammonium compounds selected from diallyldimethylammonium chloride (DADMAC), [2-(methacryloyloxy)ethyl]trimethylammonium chloride (MAOETAC), [2-(acryloyloxy)ethyl]trimethylammonium chloride (AOETAC), [2-methacrylamidoethyl]trimethylammonium chloride, [2-(acrylamido)ethyl]trimethylammonium chloride, [3-methacrylamidopropyl]trimethylammonium chloride (MAPTAC), [3-acrylamidopropyl]trimethylammonium chloride (APTAC), N-methyl-2-vinylpyridinium chloride, N-methyl-4-vinylpyridinium chloride, and N-methyl-3-vinylimidazolinium chloride.

In another particularly preferred embodiment of the invention, the structural units of component b) originate from polymerizable quaternary ammonium compounds of the above-stated structural formula (II). Preferred among these structural units of component b), in turn, are those originating from polymerizable quaternary ammonium compounds of the structural formula (II)

X⁻+NR⁷R⁸R⁹R¹⁰ (II)

in which
X⁻is a negatively charged counterion,
R⁷ is vinyl radical, allyl radical or a radical of the following structural formula (III)

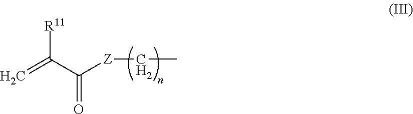

in which
R¹¹ is hydrogen or methyl,
Z is O, NH, NCH₃ or S,
n is an integer from 1 to 50,
R⁸ is a linear or branched alkyl radical having 2 to 50, preferably 2 to 25, and more preferably 4 to 8 carbon atoms, or is a radical R⁷,
R⁹ is a linear or branched alkyl radical having 1 to 50, preferably 2 to 25, and more preferably 4 to 8 carbon atoms, or is a radical R⁷, and
R¹⁰ is a linear or branched alkyl radical having 1 to 50, preferably 2 to 25, and more preferably 4 to 8 carbon atoms.

In one especially preferred embodiment of the invention, the structural units of component b) originate from polymerizable quaternary ammonium compounds selected from diallyldimethylammonium chloride (DADMAC), [2-(methacryloyloxy)-ethyl]trimethylammonium chloride (MAOETAC), [2-(acryloyloxy)ethyl]-trimethylammonium chloride (AOETAC), [2-methacrylamidoethyl]trimethylammonium chloride, [2-(acrylamido)ethyl]trimethylammonium chloride, and [3-methacrylamidopropyl]trimethylammonium chloride (MAPTAC) and [3-acryl-amidopropyl]trimethylammonium chloride (APTAC).

In an extraordinarily preferred embodiment of the invention, the polymers of the invention comprise structural units of component b) that originate from [2-(acryloyl-oxy)ethyl]trimethylammonium chloride. These polymers are preferred in oil gels, in relation to clarity and transparency, over analogous polymers comprising structural units originating from [3-methacrylamidopropyl]trimethylammonium chloride.

In a further preferred embodiment of the invention, the amount used of substances selected from monomers having more than one polymerizable group, which may have a crosslinking action (crosslinkers), for preparing the copolymers of the invention, based on the total mass of the monomers to be polymerized in the polymerization, is 0% to 20.0%, preferably 0% to 10.0%, and more preferably 0% to 5.0% by weight.

A “polymerizable group” in this context means a polymerizable double bond.

In a further preferred embodiment of the invention, the copolymers of the invention comprise one or more structural units of component c) originating from substances selected from monomers having more than one polymerizable group, which may have a crosslinking action, in an amount of 0% to 20.0%, preferably 0% to 10.0%, and more preferably 0% to 5.0% by weight, based on the total mass of the copolymers of the invention.

In a particularly preferred embodiment of the invention, the copolymers of the invention comprise no structural units of component c) which originate from substances selected from monomers having more than one polymerizable group, which may have a crosslinking action.

In a further particularly preferred embodiment of the invention, the copolymers of the invention comprise one or more structural units of component c) that originate from substances selected from monomers having more than one polymerizable group, which may have a crosslinking action.

The crosslinking structural units of component c) originate preferably from substances selected from divinylbenzene, methylenebisacrylamide (MBA), triallylamine, triallyl cyanurate, ethanediol diacrylate, ethanediol dimethacrylate (EDDMA), butanediol diacrylate, butanediol dimethacrylate (BDDMA), hexanediol diacrylate, hexanediol dimethacrylate (HDDMA), dodecanediol diacrylate, dodecanediol dimethacrylate (DDDMA), trimethylolpropane triacrylate (TMPTA), trimethylolpropane trimethacrylate (TMPTMA), glycerol diacrylate, glycerol dimethacrylate, tetraethylene glycol diacrylate, tetraethylene glycol dimethacrylate, polyethylene glycol diacrylate with molar masses between 400 and 800 g/mol, polyethylene glycol dimethacrylate with molar masses between 400 and 800 g/mol, allyl acrylate, methacrylate or ethacrylate, preferably allyl acrylate or methacrylate, and more particularly allyl methacrylate (AMA), dipropylene glycol diallyl ether, polyglycol diallyl ether, hydroquinone diallyl ether, trimethylolpropane diallyl ether, trimethylolpropane triallyl ether, tetraallyloxyethane, triethylene glycol divinyl ether, and other allyl ethers or vinyl ethers of polyfunctional alcohols, and also from mixtures of the aforesaid substances, such as, for example, from mixtures of hexanediol dimethacrylate and trimethylolpropane trimethacrylate.

Preferred among these in turn are the crosslinking structural units of component c) that originate from substances selected from methylenebisacrylamide (MBA), triallylamine, triallyl cyanurate, ethanediol dimethacrylate (EDDMA), butanediol dimethacrylate (BDDMA), hexanediol dimethacrylate (HDDMA), dodecanediol dimethacrylate (DDDMA), trimethylolpropane triacrylate (TMPTA), trimethylolpropane trimethacrylate (TMPTMA), glycerol diacrylate, polyethylene glycol diacrylate having molar masses between 400 and 800 g/mol, allyl methacrylate (AMA), and also from mixtures of the aforesaid substances, such as, for example, from mixtures of hexanediol dimethacrylate and trimethylolpropane trimethacrylate.

With particular preference the crosslinking structural units of component c) originate from hexanediol dimethacrylate, trimethylolpropane triacrylate, and mixtures of these substances.

In one especially preferred embodiment of the invention, the amount used of the crosslinker for preparing the copolymers of the invention, based on the total mass of the monomers to be polymerized in the polymerization, is 0.1% to 20.0%, preferably 0.5% to 10.0%, and are preferably 1.0% to 5.0% by weight.

Where the copolymers of the invention comprise one or more structural units of component c) that originate from substances selected from monomers having more than one polymerizable group, which may have a crosslinking action, they comprise, in a further, especially preferred embodiment of the invention, 0.1% to 20.0%, preferably 0.5% to 10.0%, and more preferably 1.0% to 5.0% by weight of these structural units, based on the total mass of the copolymers of the invention.

In a further preferred embodiment of the invention, the copolymers comprise one or more structural units of component c) that originate from substances selected from acrylic or methacrylic esters of alkyl alcohols having 1 to 5 carbon atoms, which optionally may also have been alkoxylated with 1 to 25 alkylene oxide units, such as, for example, with ethylene oxide units, with propylene oxide units or with mixtures of ethylene oxide units and propylene oxide units, preference among these compounds being given to butyl acrylate, and hydroxyethyl methacrylate (HEMA), (meth)acrylic esters of polyethylene glycols, polypropylene glycols, and polyethylene-polypropylene glycols, N-vinylpyrrolidone, and also ethene, propene, 1-butene, and 1-pentene. Among the stated compounds, butyl acrylate is preferred. Where the copolymers of the invention comprise one or more of the structural units just stated, these units are present preferably in an amount of 0.1% to 10.0% by weight, based on the total mass of the copolymer, in the copolymers of the invention.

The copolymers of the invention may also, for example, be “grafted”, meaning that they have been applied to a support. The preparation of supported polymers is general knowledge. Where a support is used, the amount of the support, based on the mass of the supported copolymer, is preferably from 0.1% to 10.0% by weight and more preferably from 2.0% to 6.0% by weight. Where a support is used, it is preferably polyvinylpyrrolidone, polyethylene glycol, polypropylene glycol or polyethylene/polypropylene glycol, and more preferably polyvinylpyrrolidone.

In the copolymers of the invention, preferably, the amount of structural units of component a) is from 60.0% to 99.5% by weight, the amount of structural units of component b) is from 0.5% to 20.0% by weight, and the amount of structural units of component c) is from 0% to 20.0% by weight.

In the copolymers of the invention, more preferably, the amount of structural units of component a) is from 75.0% to 98.5% by weight, the amount of structural units of component b) is from 0.6% to 12.5% by weight, and the amount of structural units of component c) is from 0% to 12.5% by weight.

In the copolymers of the invention, especially preferably, the amount of structural units of component a) is from 80.0% to 98.0% by weight, the amount of structural units of component b) is from 0.7% to 11.0% by weight, and the amount of structural units of component c) is from 0% to 11.0% by weight.

The ratio of the structural units originating from the compounds of the formulae (Ia), (Ib) and (Ic) within component a) of the copolymers of the invention is preferably as follows: 50.0% to 100.0% by weight of structural units originating from the compounds of the formula (Ia), 0% to 40.0% by weight of structural units originating from the compounds of the formula (Ib), and 0% to 20.0% by weight of structural units originating from the compounds of the formula (Ic). The weight ratio of structural units originating from the compound of the formula (Ia): structural units originating from the compounds of the formula (Ib): structural units originating from the compounds of the formula (Ic) in the copolymers of the invention is preferably 1.0:0 to 0.8:0 to 0.4.

In a further preferred embodiment of the invention, the copolymers of the invention comprise no structural units originating from anionic polymerizable substances.

In a further preferred embodiment of the invention, the copolymers of the invention consist of the structural units stated under components a) and b).

In a further preferred embodiment of the invention, the copolymers of the invention consist of the structural units stated under components a), b), and c). Preference in turn among these copolymers is given to those in which the structural units of component c) are selected from crosslinking structural units.

In one particularly preferred embodiment of the invention, the copolymers of the invention comprise from 95.0% to 99.0% by weight of one or more structural units originating from the compounds of the formula (Ia) of component a), preferably selected from stearyl acrylate and lauryl methacrylate, and from 0.1% to 5.0% by weight of one or more structural units originating from the compounds of component b), preferably [2-(acryloyloxy)ethyl]trimethylammonium chloride (AOETAC) (copolymers A hereinbelow).

Among the copolymers A, the copolymer consisting of 97.0% by weight of structural units originating from stearyl acrylate and 3.0% by weight of structural units originating from [2-(acryloyloxy) ethyl]trimethylammonium chloride (AOETAC) is preferred (e.g., copolymers #1, 273 and 278).

Among the copolymers A, furthermore, the copolymer consisting of 82.0% by weight of structural units originating from stearyl acrylate, 15.0% by weight of structural units originating from lauryl methacrylate, and 3.0% by weight of structural units originating from [2-(acryloyloxy)ethyl]trimethylammonium chloride (AOETAC) is preferred (e.g., copolymer #277).

In another particularly preferred embodiment of the invention, the copolymers of the invention comprise from 90.0% to 98.0% by weight of one or more structural units originating from compounds of the formula (I) of component a), preferably selected from stearyl acrylate, isostearyl acrylate, behenyl methacrylate ethoxylated with 25 EO units (B250MA) and C₂₆-C₂₈ α-olefin, from 1.5% to 5.0% by weight of one or more structural units originating from compounds of component b), preferably selected from [2-(acryloyloxy)ethyl]trimethylammonium chloride (AOETAC) and [3-methacrylamidopropyl]trimethylammonium chloride (MAPTAC) and from 0.5% to 6.0% by weight of one or more structural units originating from compounds of component c), preferably selected from crosslinkers, more particularly selected from hexanediol dimethacrylate (HDDMA) and trimethylolpropane triacrylate (TMPTA) (copolymers B hereinbelow).

Among the copolymers B, preference is given to those comprising, as structural units originating from compounds of the formula (I) of component (a), structural units originating from compounds of the formulae (Ia) and (Ib), and preference in turn is given to these in which the weight ratio of structural units originating from the compound or compounds of the formula (Ia): structural units originating from the compound or compounds of the formula (Ib)=1.0:0.1 to 1.0 (copolymers B1 hereinbelow).

Among the copolymers B1, the copolymers consisting of 56.4% by weight of structural units originating from stearyl acrylate, 39.6% by weight of structural units originating from behenyl methacrylate ethoxylated with 25 EO units (B250MA), 3.0% by weight of structural units originating from [2-(acryloyloxy)-ethyl]trimethylammonium chloride (AOETAC), and 1.0% by weight of structural units originating from hexanediol dimethacrylate (HDDMA) are preferred (e.g., copolymer #30).

Among the copolymers B1, furthermore, those copolymers consisting of 74.6% by weight of structural units originating from stearyl acrylate, 9.5% by weight of structural units originating from isostearyl acrylate, 8.6% by weight of structural units originating from behenyl methacrylate ethoxylated with 25 EO units (B250MA), 2.5% by weight of structural units originating from [2-(acryloyloxy)-ethyl]trimethylammonium chloride (AOETAC), and 4.8% by weight of structural units originating from hexanediol dimethacrylate (HDDMA) are preferred (e.g., copolymer #40).

Among the copolymers B, furthermore, preference is given to those which comprise structural units originating from compounds of the formulae (Ia), (Ib), and (Ic), and preference in turn to these in which the weight ratio of structural units originating from the compound or compounds of the formula (Ia): structural units originating from the compound or compounds of the formula (Ib): structural units originating from the compound or compounds of the formula (Ic)=10.0 to 20.0:1.0 to 3.0:1.0 (copolymers B2 hereinbelow).

Among the copolymers B2, the copolymers consisting of 78.2% by weight of structural units originating from stearyl acrylate, 9.0% by weight of structural units originating from behenyl methacrylate ethoxylated with 25 EO units (B250MA), 4.7% by weight of structural units originating from C₂₆-C₂₈ α-olefin, 3.6% by weight of structural units originating from a mixture of [2-(acryloyloxy)-ethyl]trimethylammonium chloride (AOETAC) and [3-methacrylamidopropyl]trimethylammonium chloride (MAPTAC) and 4.7% by weight of structural units originating from a mixture of hexanediol dimethacrylate (HDDMA) and trimethylolpropane triacrylate (TMPTA) are preferred (e.g., copolymer #111).

Among the copolymers B2, furthermore, the copolymers consisting of 78.2% by weight of structural units originating from stearyl acrylate, 9.0% by weight of structural units originating from behenyl methacrylate ethoxylated with 25 EO units (B250MA), 4.7% by weight of structural units originating from C₂₆-C₂₈ α-olefin, 3.6% by weight of structural units originating from [2-(acryloyloxy)-ethyl]trimethylammonium chloride (AOETAC), and 4.7% by weight of structural units originating from a mixture of hexanediol dimethacrylate (HDDMA) and trimethylolpropane triacrylate (TMPTA) are preferred (e.g., copolymer #60).

Among the copolymers B, furthermore, preference is given to those which comprise structural units originating from compounds of the formulae (Ia), (Ib), and (Ic), and preference in turn to these in which the weight ratio of structural units originating from the compound or compounds of the formula (Ia): structural units originating from the compound or compounds of the formula (Ib): structural units originating from the compound or compounds of the formula (Ic)=50.0 to 60.0:5.0 to 7.0:1.0 (copolymers B3 hereinbelow).

Among the copolymers B3, the copolymers consisting of 84.1% by weight of structural units originating from stearyl acrylate, 9.4% by weight of structural units originating from behenyl methacrylate ethoxylated with 25 EO units (B250MA), 1.6% by weight of structural units originating from C₂₆-C₂₈ α-olefin, 1.6% by weight of structural units originating from [2-(acryloyloxy)ethyl]trimethylammonium chloride (AOETAC), and 3.3% by weight of structural units originating from trimethylolpropane triacrylate (TMPTA) are preferred (e.g., copolymer #52).

The distribution of the various structural units in the copolymers of the invention may be random, blocklike, alternating or gradientlike.

The copolymers of the invention preferably possess a molecular weight of 10³ to 10⁹ g/mol, more preferably from 10⁴ to 10⁷ g/mol, and with particular preference from 5*10⁵ to 5*10⁶ g/mol.

The copolymers of the invention are prepared by radical polymerization reaction of the polymerizable substances from which the repeating structural units of components a), b), and optionally c), and also any further structural units, originate.

The invention further provides a process for preparing a copolymer of the invention, which comprises polymerizing the monomers in a radical polymerization reaction.

Serving as the polymerization medium may be all solvents which behave very largely inertly in respect of radical polymerization reactions and which allow the formulation of high molecular weights. Finding use preferably are organic solvents, more particularly lower, tertiary alcohols or hydrocarbons having 3 to 30 carbon atoms. One particularly preferred embodiment uses tert-butanol or toluene as reactive medium. Mixtures of two or more representatives from the potential solvents described are of course likewise in accordance with the invention. This also includes emulsions of mutually immiscible solvents (e.g., water/hydrocarbons). In principle, all kinds of reaction regime are suitable that lead to the polymer structures of the invention (solution polymerization, emulsion processes, precipitation processes, high-pressure processes, suspension processes, bulk polymerization, gel polymerization, and so on).

The polymerization reaction takes place preferably in the temperature range between 0 and 150° C., more preferably between 10 and 110° C., either at atmospheric pressure or under increased or reduced pressure. The polymerization may optionally also be performed under an inert gas atmosphere, preferably under nitrogen or argon.

To initiate the polymerization it is possible to use high-energy electromagnetic rays, mechanical energy, or the customary chemical polymerization initiators, such as organic peroxides, examples being benzoyl peroxide, tert.-butyl hydroperoxide, methyl ethyl ketone peroxide, cumene hydroperoxide, dilauroyl peroxide, or azo initiators, such as azodiisobutyronitrile (AIBN), azomethylbutyronitrile (AMBN), or dimethyl 2,2″-azobis(2-methylpropionate), for example.

In the course of the preparation of the copolymers of the invention it is also possible to use substances which regulate the polymerization or the chain length of the copolymers of the invention, such as methallyl sulfonate, isopropanol or dodecyl mercaptan, for example. The use of such substances in polymerization reactions is general knowledge. Where an initiator for the polymerization reaction is used, corresponding comments apply.

In the preparation of the copolymers of the invention it is also possible to use substances which control the radical polymerization by reversibly deactivating the very reactive chain ends. Where controlled radical polymerization is employed, it is carried out preferably with organic halides/transition metal complex compounds, preferably secondary alkyl halides in conjunction with copper (I) complexes, as ATRP (Atom Transfer Radical Polymerization) or through the use of linear or cyclic nitroxides, preferably in the form of what is called the TEMPO method.

One preferred embodiment of the process of the invention is characterized in that the radical polymerization takes place in an organic solvent, preferably in tert-butanol or toluene, more preferably in tert-butanol.

A further preferred embodiment of the process of the invention is characterized in that the copolymers of the invention are isolated by distillative removal of the solvent and by subsequent vacuum drying.

A further preferred embodiment of the invention is characterized in that the copolymers of the invention are isolated by precipitation in a nonsolvent, i.e., a solvent in which the copolymer of the invention is insoluble, preferably in water, and by subsequent filtration and vacuum drying.

The above-described copolymers are odor-neutral, are white to beige, and have excellent processing properties; they are kind to the skin, highly compatible with all commonplace ingredients of cosmetic formulations, and therefore suitable for preparing cosmetic, dermatological, and pharmaceutical formulations.

The invention accordingly further provides a cosmetic, pharmaceutical or dermatological formulation, preferably a cosmetic formulation, characterized in that it comprises one or more copolymers of the invention.

In one preferred embodiment of the invention, the cosmetic formulations of the invention are skin cleansing products, skincare products or hair treatment products.

The copolymers of the invention are notable for excellent oil-binding capacity and are outstandingly suitable for thickening oil-based formulations.

The invention therefore further provides for the use of one or more of the copolymers of the invention for thickening oils. These oils are preferably cosmetic oils, i.e., oils which may find use in cosmetic formulations.

The oils may advantageously be selected from the groups of triglycerides, natural and synthetic fatty substances, preferably esters of fatty acids with alcohols of low C number, such as with methanol, isopropanol, propylene glycol or glycerol, for example, or esters of fatty alcohols with alkanoic acids of low C number, or with fatty acids, or from the group of the alkylbenzoates, and also natural or synthetic hydrocarbon oils.

Substances contemplated include triglycerides of linear or branched, saturated or unsaturated, optionally hydroxylated, C₈-C₃₀ fatty acids, especially vegetable oils, such as sunflower oil, corn oil, soybean oil, rice oil, jojoba oil, babassu oil, pumpkin oil, grapeseed oil, sesame oil, walnut oil, apricot oil, orange oil, wheatgerm oil, peach kernel oil, macadamia oil, avocado oil, sweet almond oil, lady's smock oil, castor oil, olive oil, peanut oil, rapeseed oil, and coconut oil, and also synthetic triglyceride oils, examples being the commercial product Myritol® 318 and also the commercial product Velsan® CCT (caprylic/capric triglyceride, Clariant). Hydrogenated triglycerides are inventively preferred as well. It is also possible to use oils of animal origin, examples being beef tallow, perhydrosqualene, and lanolin.

A further class of preferred oily substances are the benzoic esters of linear or branched C₈₋₂₂ alkanols, examples being the commercial products Finsolv® SB (isostearyl benzoate), Finsolv® TN (C₁₂-C₁₅ alkylbenzoate), and Finsolv® EB (ethylhexyl benzoate).

A further class of preferred oily substances are the dialkyl ethers having a total of 12 to 36 carbon atoms, more particularly with 12 to 24 carbon atoms, such as, for example, di-n-octyl ether (Cetiol® OE), di-n-nonyl ether, di-n-decyl ether, di-n-undecyl ether, di-n-dodecyl ether, n-hexyl n-octyl ether, n-octyl n-decyl ether, n-decyl n-undecyl ether, n-undecyl n-dodecyl ether, and n-hexyl n-undecyl ether, di-3-ethyldecyl ether, tert-butyl n-octyl ether, isopentyl n-octyl ether, and 2-methylpentyl n-octyl ether, and also di-tert-butyl ether and diisopentyl ether.

Also contemplated are branched saturated or unsaturated fatty alcohols having 6-30 carbon atoms, isostearyl alcohol, for example, and also Guerbet alcohols.

A further class of preferred oily substances are alkyl esters of hydroxycarboxylic acids. Preferred alkyl esters of hydroxycarboxylic acids are full esters of glycolic acid, lactic acid, malic acid, tartaric acid or citric acid. Further hydroxycarboxylic esters suitable in principle are esters of β-hydroxypropionic acid, of tartronic acid, of D-gluconic acid, saccharic acid, mucic acid or glucuronic acid. Suitability as the alcohol component of these esters is possessed by primary, linear or branched aliphatic alcohols having 8 to 22 carbon atoms. Particular preference in this context is given to the esters of C₁₂-C₁₅ fatty alcohols. Esters of this type are available commercially, as for example under the trade name Cosmacol® from EniChem, Augusta Industriale.

A further class of preferred oily substances are dicarboxylic esters of linear or branched C₂-C₁₀ alkanols, such as di-n-butyl adipate (Cetiol® B), di(2-ethylhexyl) adipate, and di(2-ethylhexyl) succinate, and also diol esters such as ethylene glycol dioleate, ethylene glycol diisotridecanoate, propylene glycol di(2-ethylhexanoate), propylene glycol diisostearate, propylene glycol dipelargonate, butanediol diisostearate, and neopentyl glycol dicaprylate, and also diisotridecyl azelaate.

Preferred oily substances are also symmetrical, unsymmetrical or cyclic esters of carbonic acid with fatty alcohols, glycerol carbonate or dicaprylyl carbonate (Cetiol® CC).

A further class of preferred oily substances are the esters of dimers of unsaturated C₁₂-C₂₂ fatty acids (dimer fatty acids) with monohydric linear, branched or cyclic C₂-C₁₈ alkanols or with polyhydric linear or branched C₂-C₆ alkanols.

A further class of preferred oily substances are hydrocarbon oils, examples being those having linear or branched, saturated or unsaturated C₇-C₄₀ carbon chains, such as, for example, petrolatum, dodecane, isododecane, cholesterol, lanolin, synthetic hydrocarbons such as polyolefins, especially polyisobutene, hydrogenated polyisobutene, polydecane, and also hexadecane, isohexadecane, paraffin oils, isoparaffin oils, examples being the commercial products of the Permethyl® series, squalane, squalene, and alicyclic hydrocarbons, such as the commercial product 1,3-di(2-ethylhexyl)cyclohexane (Cetiol® S), for example, ozokerite, and ceresin.

Silicone oils and silicone waxes are likewise contemplated, preferably dimethylpolysiloxanes and cyclomethicones, polydialkylsiloxanes R₃SiO(R₂SiO)ₓSiR₃, where R is methyl or ethyl, more preferably methyl, and x is a number from 2 to 500, examples being the dimethicones available under the trade names VICASIL (General Electric Company), DOW CORNING 200, DOW CORNING 225, DOW CORNING 200 (Dow Corning Corporation), and also the dimethicones available as SilCare® Silicone 41M65, SilCare® Silicone 41M70, SilCare® Silicone 41M80 (Clariant), stearyldimethylpolysiloxane, C₂₀-C₂₄-alkyl-dimethylpolysiloxane, C₂₄-C₂₈-alkyl-dimethylpolysiloxane, and also the methicones available as SilCare® Silicone 41M40 and SilCare® Silicone 41M50 (Clariant), and additionally trimethylsiloxysilicates [(CH₂)₃SiO)_{1/2}]ₓ[SiO₂]_{y}, where x is a number from 1 to 500 and y is a number from 1 to 500, dimethiconols R₃SiO[R₂SiO]ₓSiR₂OH and HOR₂SiO[R₂SiO]ₓSiR₂OH, where R is methyl or ethyl and x is a number up to 500, polyalkylarylsiloxanes, examples being the polymethylphenylsiloxanes available under the commercial designations SF 1075 METHYLPHENYL FLUID (General Electric Company) and 556 COSMETIC GRADE PHENYL TRIMETHICONE FLUID (Dow Corning Corporation), polydiarylsiloxanes, silicone resins, cyclic silicones, and amino-, fatty acid-, alcohol-, polyether-, epoxy-, fluorine- and/or alkyl-modified silicone compounds, and also polyethersiloxane copolymers.

The copolymers of the invention can also be used advantageously for preparing oil gels. In a further preferred embodiment of the invention, therefore, the cosmetic, pharmaceutical or dermatological formulations of the invention are oil gels such as thickened baby oils, for example.

The oil gels of the invention comprise preferably 90.0% to 99.99% by weight, more preferably 94.0% to 99.9% by weight and with particular preference 97.0% to 99.5% by weight of one or more oils or oily substances, and preferably 0.01% to 10.0% by weight, more preferably 0.1% to 6.0% by weight, and with particular preference 0.5% to 3.0% by weight of one or more of the copolymers of the invention, in each case based on the overall oil gel. The oils or oily substances preferably present in the oil gels of the invention correspond to the above-identified preferred oils or oily substances.

Further advantageous are the outstanding conditioning properties of the copolymers of the invention with respect to keratinic fibers. They produce an excellent skin feel and are superior to the wax-containing formulations, which frequently exhibit a sticky and dull behavior. They enhance the shine of the hair, and also its easy combability.

The present invention accordingly further provides for the use of one or more of the copolymers of the invention for conditioning fibers, preferably keratinic fibers, more preferably in cosmetic formulations.

One particularly preferred embodiment of the invention sees the cosmetic formulations of the invention as hair treatment compositions. Preference here is given to hair treatment compositions, more particularly hair conditioning products such as cream washes and hair rinses, which are used for improving the shine and combability of the hair and for protecting it from heat. In an especially preferred embodiment of the invention, the hair treatment compositions of the invention, more particularly the hair treatment compositions just identified, comprise one or more copolymers of the invention and one or more further cationic surfactants such as cetrimonium chloride, behentrimonium chloride, stearamidopropyldimethylamine, behenamidopropyldimethylamine or quaternary ammonium compounds with an ester basis, and/or one or more silicones.

On account of their substantivity and low water-solubility, the copolymers of the invention are suitable for application as water resistance improvers. The invention accordingly further provides for the use of one or more of the polymers of the invention as water resistance improvers.

The copolymers of the invention are suitable, furthermore, for the surface treatment of pigments such as FeO, TiO₂, and ZnO, for example. The invention accordingly further provides for the use of one or more of the polymers of the invention for surface treatment of pigments.

The copolymers of the invention are advantageously suitable, moreover, for enhancing the water resistance of sun protection formulations.

The invention accordingly further provides for the use of one or more of the copolymers of the invention for enhancing the water resistance of sun protection formulations.

Furthermore, the copolymers of the invention are advantageously suitable for increasing the sun protection factor in sun protection formulations.

The invention accordingly further provides for the use of one or more of the copolymers of the invention for increasing the sun protection factor of sun protection formulations.

In a further preferred embodiment of the invention, the formulations of the invention are sun protection compositions and comprise one or more organic and/or inorganic sun protection filters.

On account of the pigment-dispersing properties of the copolymers of the invention, the cosmetic formulations or products of the invention, in a further preferred embodiment of the invention, are decorative cosmetics such as, for example, powders, foundations, mascaras or lipsticks.

The copolymers of the invention are additionally suitable advantageously as emulsifier, particularly in cosmetic formulations.

The invention therefore further provides for the use of one or more of the copolymers of the invention as emulsifier, preferably in cosmetic formulations.

In a further preferred embodiment of the invention, the formulations of the invention are in the form of emulsions of the water-in-oil or oil-in-water type, especially the skin care products of the invention. In a particularly preferred embodiment of the invention, besides the one or more copolymers of the invention, these emulsions comprise one or more nonionic emulsifiers. Among the emulsions, preference is given to the emulsions of the water-in-oil type.

A further preferred embodiment of the invention is the use of one or more of the copolymers in formulations which are free from wax components.

The copolymers of the invention are suitable not only as thickeners, consistency modifiers and sensorial additives but also as emulsifiers, solubilizers, dispersants, suspension media, lubricants, adherents, and stabilizers.

The invention accordingly further provides for the use of one or more of the copolymers of the invention as thickeners, consistency modifiers, sensorial additives, emulsifiers, solubilizers, dispersants, suspension media, lubricants, adherents or stabilizers.

Relative to the completed cosmetic, pharmaceutical or dermatological formulations, the formulations of the invention comprise preferably 0.01% to 10.0% by weight, more preferably 0.1% to 6.0% by weight, and with particular preference 0.5% to 3.0% by weight of the copolymers of the invention.

The formulations of the invention may comprise cationic, nonionic, ampholytic surfactants, betaine surfactants and/or, in small amounts, anionic surfactants.

The total amount of the surfactants used in the compositions of the invention (e.g., in the case of rinse-off products), based on the completed formulations of the invention, is preferably from 1.0% to 70.0% by weight, more preferably from 5.0% to 40.0% by weight, and with particular preference from 10.0% to 35.0% by weight.

Preferred anionic surfactants are (C₁₀-C₂₂)-alkyl carboxylates and alkylene carboxylates, alkyl ether carboxylates, fatty alcohol sulfates, fatty alcohol ether sulfates, alkylamide sulfates and alkylamide sulfonates, fatty acid alkylamide polyglycol ether sulfates, alkanesulfonates and hydroxyalkanesulfonates, olefinsulfonates, acyl esters of isethionates, α-sulfo fatty acid esters, alkylbenzenesulfonates, alkylphenol glycol ether sulfonates, sulfosuccinates, sulfosuccinic monoesters and diesters, fatty alcohol phosphates, fatty alcohol ether phosphates, protein-fatty acid condensates, alkylmonoglyceride sulfates and alkylmonoglyceride sulfonates, alkylglyceride ether sulfonates, fatty acid methyltaurides, fatty acid sarcosinates, sulforicinoleates, acylglutamates and acylglycinates. These compounds and their mixtures are used in the form of their water-soluble or water-dispersible salts, examples being the sodium, potassium, magnesium, ammonium salts and mono-, di- and triethanolammonium and analogous alkylammonium salts.

The amount of the anionic surfactants in the formulations of the invention is preferably from 0.1% to 10.0% by weight, more preferably from 0.2% to 5.0% by weight, and with particular preference from 0.5% to 2.0% by weight, based on the completed formulations.

Preferred cationic surfactants are quaternary ammonium salts, such as di-(C₈-C₂₂)-alkyl-dimethylammonium chloride or bromide, preferably di-(C₈-C₂₂)-alkyl-dimethylammonium chloride or bromide; (C₈-C₂₂)-alkyl-dimethylethylammonium chloride or bromide; (C₈-C₂₂)-alkyl-trimethylammonium chloride or bromide, preferably cetyltrimethylammonium chloride or bromide and (C₈-C₂₂)-alkyl-trimethylammonium chloride or bromide; (C₁₀-C₂₄)-alkyl-dimethylbenzylammonium chloride or bromide, preferably (C₁₂-C₁₈)-alkyl-dimethylbenzylammonium chloride, (C₈-C₂₂)-alkyl-dimethyl-hydroxyethylammonium chloride, phosphate, sulfate, and lactate, (C₈-C₂₂)-alkylamidopropyltrimethylammonium chloride and methosulfate, N,N-bis(2-C₈-C₂₂-alkanoyl-oxyethyl)-dimethylammonium chloride and methosulfate and N,N-bis(2-C₈-C₂₂-alkanoyl-oxyethyl)hydroxyethyl-methyl-ammonium chloride and methosulfate.

The amount of the cationic surfactants in the formulations of the invention is preferably from 0.1% to 10.0% by weight, more preferably from 0.5% to 7.0% by weight, and with particular preference from 1.0% to 5.0% by weight, based on the completed formulations.

Preferred nonionic surfactants are fatty alcohol ethoxylates (alkylpolyethylene glycols); alkylphenolpolyethylene glycols; fatty amine ethoxylates (alkylaminopolyethylene glycols); fatty acid ethoxylates (acylpolyethylene glycols); polypropylene glycol ethoxylates (Pluronics®); fatty acid alkanolamides (fatty acid amide polyethylene glycols); sucrose esters; sorbitol esters and sorbitan esters and their polyglycol ethers, and also C₈-C₂₂-alkylpolyglucosides.

The amount of the nonionic surfactants in the formulations of the invention (in the case of rinse-off products, for example) is preferably in the range from 1.0% to 20.0% by weight, more preferably from 2.0% to 10.0% by weight, and with particular preference from 3.0% to 7.0% by weight, based on the completed formulations.

The formulations of the invention may further comprise amphoteric surfactants. These surfactants may be described as derivatives of long-chain secondary or tertiary amines which possess an alkyl group having 8 to 18 carbon atoms and in which a further group is substituted by an anionic group which imparts solubility in water, as for example by a carboxyl, sulfate or sulfonate group. Preferred amphoteric surfactants are N—(C₁₂-C₁₈)-alkyl-β-aminopropionates and N—(C₁₂-C₁₈)-alkyl-β-iminodipropionates in the form of alkali metal salts and mono-, di-, and trialkylammonium salts. Suitable further surfactants are also amine oxides. These are oxides of tertiary amines with a long-chain group of 8 to 18 carbon atoms and two usually short-chain alkyl groups with 1 to 4 carbon atoms. Preference is given here, for example, to the C₁₀ to C₁₈ alkyl-dimethylamine oxides, fatty acid amidoalkyldimethylamine oxide.

A further preferred group of surfactants are betaine surfactants, also called zwitterionic surfactants. These surfactants contain in the same molecule a cationic group, more particularly an ammonium group, and an anionic group, which may be a carboxylate group, sulfate group or sulfonate group. Suitable betaines are preferably alkyl betaines such as coco-betaine or fatty acid alkylamidopropyl betaines, examples being cocoacylamidopropyldimethyl betaine, or the C₁₂ to C₁₈ dimethylaminohexanoates and/or the C₁₀ to C₁₈ acylamidopropanedimethyl betaines.

As further auxiliaries and additives, the formulations of the invention may comprise waxes, emulsifiers, co-emulsifiers, solubilizers, electrolytes, hydroxyl acids, stabilizers, further cationic polymers, film formers, further thickeners, gelling agents, superfatting agents, refatting agents, antimicrobial actives, biogenic actives, astringents, deodorants, non protection antioxidants, humectants, solvents, filters, colorants, fragrances, pearlescents, opacifiers and/or water-soluble silicones.

The amount of the amphoteric surfactants and/or betaine surfactants in the formulations of the invention is preferably from 0.5% to 20.0% by weight and more preferably from 1.0% to 10.0% by weight, based on the completed formulations.

Preferred surfactants are lauryl sulfate, laureth sulfate, cocoamidopropyl betaine, alkyl betaines such as, for example, coco-betaine, sodium cocoylglutamate, and lauroamphoacetate.

In a further preferred embodiment of the invention the formulations of the invention further comprise, as foam boosters, co-surfactants from the group of alkyl betaines, alkylamido betaines, aminopropionates, aminoglycinates, imidazolinium betaines, and sulfo betaines, amine oxides, fatty acid alkanolamides, and polyhydroxyam ides.

As further auxiliaries and additives, the formulations of the invention may comprise waxes, emulsifiers, co-emulsifiers, solubilizers, electrolytes, hydroxyl acids, stabilizers, further cationic polymers, film formers, further thickeners, gelling agents, superfatting agents, refatting agents, antimicrobial actives, biogeneic actives, astringents, deodorants, unprotection filters, antioxidants, humectants, solvents, colorants, fragrances, pearlescents, opacifiers and/or water-soluble silicones.

The formulations of the invention may comprise waxes, examples being paraffin waxes, microwaxes, and ozokerites, beeswax and its component fractions, and also beeswax derivatives, waxes from the group of homopolymeric polyethylenes or copolymers of α-olefins, and also natural waxes such as rice wax, candelilla wax, carnauba wax, Japan wax or shellac wax.

Emulsifiers, co-emulsifiers and solubilizers may be nonionic, anionic, cationic or amphoteric surface-active compounds.

Suitable nonionic surface-active compounds are preferably as follows:

Adducts of 0 to 30 mol of ethylene oxide and/or 0 to 5 mol of propylene oxide with linear fatty alcohols having 8 to 22 carbon atoms, with fatty acids having 12 to 22 carbon atoms, with alkylphenols having 8 to 15 carbon atoms in the alkyl group, and with sorbitan and/or sorbitol esters; (C₁₂-C₁₈) fatty acid monoesters and diesters of adducts of 0 to 30 mol of ethylene oxide with glycerol; glycerol mono esters and diesters and sorbitan monoesters and diesters of saturated and unsaturated fatty acids having 6 to 22 carbon atoms and, if desired, their ethylene oxide adducts; adducts of 15 to 60 mol of ethylene oxide with castor oil and/or hydrogenated castor oil; polyol esters and especially polyglycerol esters, such as polyglycerol polyricinoleate and polyglycerol poly-12-hydroxystearate, for example. Likewise suitable with preference are ethoxylated fatty amines, fatty acid amides, fatty acid alkanolamides, and mixtures of compounds from two or more of these classes of substance.

Examples of suitable ionogenic co-emulsifiers include anionic emulsifiers, such as mono-, di- or tri-phosphoric esters, soaps (e.g., sodium stearate), fatty alcohol sulfates, and also cationic emulsifiers such as mono-, di-, and tri-alkyl quats and their polymeric derivatives.

Amphoteric emulsifiers preferentially available are alkylaminoalkylcarboxylic acids, betaines, sulfo betaines, and imidazoline derivatives.

Used with particular preference are fatty alcohol ethoxylates selected from the group of ethoxylated stearyl alcohols, isostearyl alcohols, cetyl alcohols, isocetyl alcohols, oleyl alcohols, lauryl alcohols, isolauryl alcohols, and cetylstearyl alcohols, more particularly polyethylene glycol(13) stearyl ether, polyethylene glycol(14) stearyl ether, polyethylene glycol(15) stearyl ether, polyethylene glycol(16) stearyl ether, polyethylene glycol(17) stearyl ether, polyethylene glycol(18) stearyl ether, polyethylene glycol(19) stearyl ether, polyethylene glycol(20) stearyl ether, polyethylene glycol(12) isostearyl ether, polyethylene glycol(13) isostearyl ether, polyethylene glycol(14) isostearyl ether, polyethylene glycol(15) isostearyl ether, polyethylene glycol(16) isostearyl ether, polyethylene glycol(17) isostearyl ether, polyethylene glycol(18) isostearyl ether, polyethylene glycol(19) isostearyl ether, polyethylene glycol(20) isostearyl ether, polyethylene glycol(13) cetyl ether, polyethylene glycol(14) cetyl ether, polyethylene glycol(15) cetyl ether, polyethylene glycol(16) cetyl ether, polyethylene glycol(17) cetyl ether, polyethylene glycol(18) cetyl ether, polyethylene glycol(19) cetyl ether, polyethylene glycol(20) cetyl ether, polyethylene glycol(13) isocetyl ether, polyethylene glycol(14) isocetyl ether, polyethylene glycol(15) isocetyl ether, polyethylene glycol(16) isocetyl ether, polyethylene glycol(17) isocetyl ether, polyethylene glycol(18) isocetyl ether, polyethylene glycol(19) isocetyl ether, polyethylene glycol(20) isocetyl ether, polyethylene glycol(12) oleyl ether, polyethylene glycol(13) oleyl ether, polyethylene glycol(14) oleyl ether, polyethylene glycol(15) oleyl ether, polyethylene glycol(12) lauryl ether, polyethylene glycol(12) isolauryl ether, polyethylene glycol(13) cetylstearyl ether, polyethylene glycol(14) cetylstearyl ether, polyethylene glycol(15) cetylstearyl ether, polyethylene glycol(16) cetylstearyl ether, polyethylene glycol(17) cetylstearyl ether, polyethylene glycol(18) cetylstearyl ether, polyethylene glycol(19) cetylstearyl ether.

Fatty acid ethoxylates selected from the group of ethoxylated stearates, isostearates and oleates, in particular polyethylene glycol(20) stearate, polyethylene glycol(21) stearate, polyethylene glycol(22) stearate, polyethylene glycol(23) stearate, polyethylene glycol(24) stearate, polyethylene glycol(25) stearate, polyethylene glycol(12) isostearate, polyethylene glycol(13) isostearate, polyethylene glycol(14) isostearate, polyethylene glycol(15) isostearate, polyethylene glycol(16) isostearate, polyethylene glycol(17) isostearate, polyethylene glycol(18) isostearate, polyethylene glycol(19) isostearate, polyethylene glycol(20) isostearate, polyethylene glycol(21) isostearate, polyethylene glycol(22) isostearate, polyethylene glycol(23) isostearate, polyethylene glycol(24) isostearate, polyethylene glycol(25) isostearate, polyethylene glycol(12) oleate, polyethylene glycol(13) oleate, polyethylene glycol(14) oleate, polyethylene glycol(15) oleate, polyethylene glycol(16) oleate, polyethylene glycol(17) oleate, polyethylene glycol(18) oleate, polyethylene glycol(19) oleate, polyethylene glycol(20)oleate are likewise preferred.

Sodium laureth-11 carboxylate can advantageously be used as ethoxylated alkylether carboxylic acid or salts thereof.

Ethoxylated triglycerides which can be used are advantageously polyethylene glycol(60) evening primrose glycerides.

It is furthermore advantageous to select the polyethylene glycol glycerol fatty acid esters from the group polyethylene glycol(20) glyceryl laurate, polyethylene glycol(6) glyceryl caprate/caprinate, polyethylene glycol(20) glyceryl oleate, polyethylene glycol(20) glyceryl isostearate, and polyethylene glycol(18) glyceryl oleate/cocoate.

Among the sorbitan esters, polyethylene glycol(20) sorbitan monolaurate, polyethylene glycol(20) sorbitan monostearate, polyethylene glycol(20) sorbitan monoisostearate, polyethylene glycol(20) sorbitan monopalmitate, polyethylene glycol(20) sorbitan monooleate are particularly suitable.

Particularly advantageous co-emulsifiers are glyceryl monostearate, glyceryl monooleate, diglyceryl monostearate, glyceryl isostearate, polyglyceryl-3 oleate, polyglyceryl-3 diisostearate, polyglyceryl-4 isostearate, polyglyceryl-2 dipolyhydroxystearate, polyglyceryl-4 dipolyhydroxystearate, PEG-30 dipolyhydroxystearate, diisostearoyl polyglyceryl-3 diisostearate, glycol distearate and polyglyceryl-3 dipolyhydroxystearate, sorbitan monoisostearate, sorbitan stearate, sorbitan oleate, sucrose distearate, lecithin, PEG-7-hydrogenated castor oil, cetyl alcohol, stearyl alcohol, behenyl alcohol, isobehenyl alcohol and polyethylene glycol(2) stearyl ether (steareth-2), alkylmethicone copolyols, and alkyldimethicone copolyols, in particular cetyldimethicone copolyol, laurylmethiconecopolyol.

The formulations of the invention can comprise one or more of the emulsifiers, co-emulsifiers or solubilizers in amounts of from 0.1% to 20.0% by weight, preferably 1.0% to 15.0% by weight, and more preferably 3.0% to 10.0% by weight, based on the completed formulations.

As electrolyte use may be made of inorganic salts, preferably ammonium or metal salts, particularly preferably of halides, among them in turn preferably CaCl₂, MgCl₂, LiCl, KCl, NaCl, carbonates, hydrogencarbonates, phosphates, sulfates, nitrates, especially preferably sodium chloride, and/or organic salts, preferably ammonium or metal salts, particularly preferably of glycolic acid, lactic acid, citric acid, tartaric acid, mandelic acid, salicylic acid, ascorbic acid, pyruvic acid, fumaric acid, retinoic acid, sulfonic acids, benzoic acid, kojic acid, fruit acid, malic acid, gluconic acid or galacturonic acid.

Included among these salts are aluminum salts, preferably aluminum chlorohydrate or aluminum-zirconium complex salts.

As electrolyte, the formulations of the invention can also comprise mixtures of different salts. The content of the one or more electrolytes, based on the total formulation of the invention, is preferably from 0.1% to 20.0% by weight, more preferably from 0.2% to 10.0% by weight, and especially preferably from 0.5% to 5.0% by weight.

In terms of hydroxy acids, the formulations of the invention may comprise preferably lactic acid, glycolic acid, salicylic acid, citric acid or polyglycol diacids in free or partial neutralization. Furthermore, formulations of the invention comprising vitamin C or vitamin C derivatives, dihydroxyacetone or skin-whitening actives such as arbutin or glycyrrhetinic acid and salts thereof may be stabilized. The amount of one or more of these substances just stated, based on total formulation of the invention, is preferably from 0.1% to 20.0% by weight, more preferably from 0.2% to 10.0% by weight, and with particular preference from 0.5% to 5.0% by weight.

For the copolymers of the invention it is possible, as additional stabilizers to use metal salts of fatty acids, such as, for example, magnesium stearate, aluminum stearate and/or zinc stearate, preferably in amounts of from 0.1% to 10.0% by weight, preferably from 0.5% to 8.0% by weight, and more preferably from 1.0% to 5.0% by weight, based on the completed formulations.

Suitable cationic polymers are those known under the INCI name “Polyquaternium”, in particular Polyquaternium-31, Polyquaternium-16, Polyquaternium-24, Polyquaternium-7, Polyquaternium-22, Polyquaternium-39, Polyquaternium-28, Polyquaternium-2, Polyquaternium-10, Polyquaternium-11, and Polyquaternium 37&mineral oil&PPG trideceth (Salcare SC₉₅), PVP-dimethylaminoethyl methacrylate copolymer, guar hydroxypropyltriammonium chloride, and calcium alginate and ammonium alginate. Furthermore, cationic cellulose derivatives; cationic starch; copolymers of diallylammonium salts and acrylamides; quaternized vinylpyrrolidone/vinylimidazole polymers; condensation products of polyglycols and amines; quaternized collagen polypeptides; quaternized wheat polypeptides; polyethyleneimines; cationic silicone polymers, such as, for example, amidomethicones; copolymers of adipic acid and dimethylaminohydroxypropyldiethylenetriamine; polyaminopolyamide and cationic chitin derivatives, such as, for example, chitosan, can be used.

The formulations of the invention can comprise one or more of the aforementioned cationic polymers in amounts of from 0.1% to 5.0% by weight, preferably from 0.2% to 3.0% by weight, more preferably from 0.5% to 2.0% by weight, based on the completed formulations.

Furthermore, the formulations of the invention can comprise film formers which, depending on the intended use, are selected from salts of phenylbenzimidazolesulfonic acid, water-soluble polyurethanes, for example C₁₀-polycarbamyl polyglyceryl ester, polyvinyl alcohol, polyvinylpyrrolidone copolymers, for example vinylpyrrolidone/vinyl acetate copolymer, water-soluble acrylic acid polymers/copolymers and esters or salts thereof, for example partial ester copolymers of acrylic acid/methacrylic acid, water-soluble cellulose, for example hydroxymethylcellulose, hydroxyethylcellulose, hydroxypropylcellulose, water-soluble quaterniums, polyquaterniums, carboxyvinyl polymers, such as carbomers and salts thereof, polysaccharides, for example polydextrose and glucan, vinyl acetate/crotonate, for example available under the trade name Aristoflex® A 60 (Clariant).

The formulations of the invention can comprise one or more film formers in amounts of from 0.1% to 10.0% by weight, preferably from 0.2% to 5.0% by weight, and more preferably from 0.5% to 3.0% by weight, based on the completed formulations.

The desired viscosity of the formulations can be established by adding further thickeners and gelling agents. Suitable are preferably cellulose ethers and other cellulose derivatives (e.g., carboxymethylcellulose, hydroxyethylcellulose), gelatin, starch and starch derivatives, sodium alginates, fatty acid polyethylene glycol esters, agar agar, tragacanth or dextrin derivatives, in particular dextrin esters. Furthermore, metal salts of fatty acids, preferably having 12 to 22 carbon atoms, for example sodium stearate, sodium palmitate, sodium laurate, sodium arachidates, sodium behenate, potassium stearate, potassium palmitate, sodium myristate, aluminum monostearate, hydroxyl fatty acids, for example 12-hydroxystearic acid, 16-hydroxyhexadecanoyl acid; fatty acid amides; fatty acid alkanolamides; dibenzalsorbitol and alcohol-soluble polyamides and polyacrylamides or mixtures of such are suitable. Furthermore, crosslinked and uncrosslinked polyacrylates such as carbomers, sodium polyacrylates or polymers containing sulfonic acid, such as ammonium acryloyldimethyltaurate/VP copolymer, can be used.

In one further preferred embodiment of the invention, the formulations of the invention contain 0.01% to 20.0% by weight, more preferably 0.1% to 10.0% by weight, with particular preference 0.2% to 3.0% by weight, and very preferably 0.4% to 2.0% by weight, of thickeners and/or gelling agents, based on the completed formulations of the invention.

Superfatting agents which can be used are preferably lanolin and lecithin, nonethoxylated and polyethoxylated or acylated lanolin derivatives and lecithin derivatives, polyol fatty acid esters, mono-, di- and triglycerides and/or fatty acid alkanolamides, where the latter simultaneously serve as foam stabilizers, and which are preferably used in amounts of from 0.01% to 10.0% by weight, more preferably from 0.1% to 5.0% by weight and especially preferably from 0.5% to 3.0% by weight, based on the completed formulations of the invention.

Active antimicrobial ingredients that can be used are cetyltrimethylammonium chloride, cetylpyridinium chloride, benzethonium chloride, diisobutylethoxyethyldimethylbenzylammonium chloride, sodium aluminum chlorohydroxylactate, triethyl citrate, tricetylmethylammonium chloride, 2,4,4′-trichloro-2′-hydroxydiphenyl ether (triclosan), phenoxyethanol, 1,5-pentanediol, 1,6-hexanediol, 3,4,4′-trichlorocarbanilide (triclocarban), diaminoalkylamide, for example L-lysine hexadecylamide, citrate heavy metal salts, salicylates, piroctoses, in particular zinc salts, pyrithiones and heavy metal salts thereof, in particular zinc pyrithione, zinc phenol sulfate, farnesol, ketoconazole, oxiconazole, bifonazole, butoconazole, cloconazole, clotrimazole, econazole, enilconazole, fenticonazole, isoconazole, miconazole, sulconazole, tioconazole, fluconazole, itraconazole, terconazole, naftifine and terbinafine, selenium disulfide and Octopirox®, iodopropynyl butylcarbamate, methylchloroisothiazolinone, methylisothiazolinone, methyldibromoglutaronitrile, AgCl, chloroxylenol, Na salt of diethylhexyl sulfosuccinate, sodium benzoate, and phenoxyethanol, benzyl alcohol, phenoxyisopropanol, parabens, preferably butyl, ethyl, methyl and propyl paraben, and Na salts thereof, pentanediol, 1,2-octanediol, 2-bromo-2-nitropropane-1,3-diol, ethylhexylglycerol, benzyl alcohol, sorbic acid, benzoic acid, lactic acid, imidazolidinylurea, diazolidinylurea, dimethyloldimethylhydantoin (DMDMH), Na salt of hydroxymethylglycine, and combinations of these active substances.

The formulations of the invention comprise the active antimicrobial ingredients preferably in amounts from 0.001% to 5.0% by weight, more preferably from 0.01% to 3.0% by weight, and with particular preference from 0.1% to 2.0% by weight, based on the completed formulations of the invention.

The formulations of the invention may further comprise active biogenic ingredients selected from plant extracts, such as, for example, aloe vera, and also local anesthetics, antibiotics, antiphlogistics, antiallergics, corticosteroids, sebostatics, Bisabolol®, allantoin, Phytantriol®, proteins, vitamins selected from niacin, biotin, vitamin B2, vitamin B3, vitamin B6, vitamin B3 derivatives (salts, acids, esters, amides, alcohols), vitamin C and vitamin C derivatives (salts, acids, esters, amides, alcohols), preferably as sodium salt of the monophosphoric acid ester of ascorbic acid or as magnesium salt of the phosphoric acid ester of ascorbic acid, tocopherol and tocopherol acetate, and also vitamin E and/or derivatives thereof.

The formulations of the invention may comprise active biogenic ingredients preferably in amounts from 0.001% to 5.0% by weight, more preferably from 0.01% to 3.0% by weight, and with particular preference from 0.1% to 2.0% by weight, based on the completed formulations.

The formulations of the invention can comprise astringents, preferably magnesium oxide, aluminum oxide, titanium dioxide, zirconium dioxide and zinc oxide, oxide hydrates, preferably aluminum oxide hydrate (boehmite) and hydroxides, preferably of calcium, magnesium, aluminum, titanium, zirconium or zinc, and also aluminum chlorohydrates, preferably in amounts of from 0% to 50.0% by weight, particularly preferably in amounts of from 0.01% to 10.0% by weight and especially preferably in amounts of from 0.1% to 10.0% by weight. Allantoin and bisabolol are preferred as deodorizing substances. They are preferably used in amounts of from 0.0001% to 10.0% by weight.

The formulations of the invention can comprise microfine titanium dioxide, mica-titanium oxide, iron oxides, mica-iron oxide, zinc oxide, silicon oxides, ultramarine blue, chromium oxides as pigments/micropigments and also as inorganic sun protection filters.

The formulations of the invention may comprise one or more organic sun protection filters, preferably selected from 4-aminobenzoic acid, 3-(4′-trimethylammonium)benzylideneboran-2-one methyl sulfate, camphor benzalkoniummethosulfate, 3,3,5-trimethylcyclohexyl salicylate, 2-hydroxy-4-methoxybenzophenone, 2-phenylbenzimidazole-5-sulfonic acid and its potassium, sodium and triethanolamine salts, 3,3′-(1,4-phenylenedimethine)bis(7,7-dimethyl-2-oxobicyclo[2.2.1]heptane-1-methanesulfonic acid) and its salts, 1-(4-tert-butylphenyl)-3-(4-methoxyphenyl)propane-1,3-dione, 3-(4′-sulfo)benzylidenebornan-2-one and its salts, 2-ethylhexyl 2-cyano-3,3-diphenylacrylate, polymers of N-[2(and 4)-(2-oxoborn-3-ylidenemethyl)benzyl]acrylamide, 2-ethylhexyl 4-methoxycinnamate, ethoxylated ethyl 4-aminobenzoate, isoamyl 4-methoxycinnamate, 2,4,6-tris[p-(2-ethyl hexyloxycarbonyl)anilino]-1,3,5-triazine, 2-(2H-benzotriazol-2-yl)-4-methyl-6-(2-methyl-3-(1,3,3,3-tetramethyl-1-(trimethylsilyloxy)disiloxanyl)propyl)phenol, bis(2-ethylhexyl) 4,4′-[(6-[4-((1,1-dimethylethyl)aminocarbonyl)phenylamino]-1,3,5-triazin-2,4-yl)diimino]bisbenzoate, benzophenone-3, benzophenone-4 (acid), 3-(4′-methylbenzylidene)-DL-camphor, 3-benzylidenecamphor, 2-ethylhexyl salicylate, 2-ethylhexyl 4-dimethylaminobenzoate, hydroxy-4-methoxybenzophenone-5-sulfon ic acid (sulfisobenzone) and the sodium salt, 4-isopropylbenzyl salicylate, N,N,N-trimethyl-4-(2-oxoborn-3-ylidenemethyl)anilium methyl sulfate, homosalate (INN), oxybenzone (INN), 2-phenylbenzimidazole-5-sulfonic acid and its sodium, potassium and triethanolamine salts, octylmethoxycinnamic acid, isopentyl-4-methoxycinnamic acid, isoamyl-p-methoxycinnamic acid, 2,4,6-trianilino(p-carbo-2′-ethylhexyl-1′-oxy)-1,3,5-triazine (octyltriazone) phenol, 2-2(2H-benzotriazol-2-yl)-4-methyl-6-(2-methyl-3-(1,3,3,3-tetramethyl-1-(trimethylsilyl)oxy)disiloxanyl)propyl (drometrizoletrisiloxane) benzoic acid, 4,4-((6-(((1,1-dimethylethyl)amino)carbonyl)phenyl)amino)-1,3,5-triazine-2,4-diyl)diimino)bis,bis(2-ethylhexyl) ester)benzoic acid, 4,4-((6-(((1,1-dimethylethyl)amino)carbonyl)phenyl)amino)-1,3,5-triazine-2,4-diyl)diimino)bis,bis(2-ethylhexyl)ester), 3-(4′-methylbenzylidene)-DL-camphor (4-methylbenzylidenecamphor), benzylidenecamphorsulfonic acid, octocrylene, polyacrylamidomethylbenzylidenecamphor, 2-ethylhexyl salicylate (octylsalicylate), ethyl-2-hexyl 4-dimethylaminobenzoate (octyldimethyl PABA), PEG-25 PABA, 2-hydroxy-4-methoxybenzophenone-5-sulfonic acid (benzophenone-5) and the Na salt, 2,2′-methylenebis-6-(2H-benzotriazol-2-yl)-4-(tetramethylbutyl)-1,1,3,3-phenol, sodium salt of 2-2′-bis(1,4-phenylene)-1H-benzimidazole-4,6-disulfonic acid, (1,3,5)-triazine-2,4-bis((4-(2-ethylhexyloxy)-2-hydroxy)phenyl)-6-(4-methoxyphenyl), 2-ethylhexyl 2-cyano-3,3-diphenyl-2-propenoate, glyceryl octanoate, di-p-methoxycinnamic acid, p-aminobenzoic acid and esters thereof, 4-tert-butyl-4′-methoxydibenzoylmethane, 4-(2-β-glucopyranoxy)propoxy-2-hydroxybenzophenone, octyl salicylate, methyl-2,5-diisopropylcinnamic acid, cinoxate, dihydroxydimethoxybenzophenone, disodium salt of 2,2′-dihydroxy-4,4′-dimethoxy-5,5′-d isulfobenzophenone, dihydroxybenzophenone, 1,3,4-dimethoxyphenyl-4,4-dimethyl-1,3-pentanedione, 2-ethylhexyl dimethoxybenzyl idened ioxoim idazol id inepropionate, methylenebisbenzotriazolyl tetramethylbutylphenol, phenyl dibenzimidazoletetrasulfonate, bis-ethylhexyloxyphenol methoxyphenol triazine, tetrahydroxybenzophenones, terephthalylidenedicamphorsulfonic acid, 2,4,6-tris[4,2-ethylhexyloxy carbonyl)anilino]-1,3,5-triazine, methylbis(trimethylsiloxy)silylisopentyltrimethoxycinnamic acid, amyl p-dimethylaminobenzoate, amyl p-dimethylaminobenzoate, 2-ethylhexyl p-dimethylaminobenzoate, isopropyl-p-methoxycinnamic acid/diisopropylcinnamic acid esters, 2-ethylhexyl-p-methoxycinnamic acid, 2-hydroxy-4-methoxybenzophenone, 2-hydroxy-4-methoxybenzophenone-5-sulfonic acid and the trihydrate, and also 2-hydroxy-4-methoxybenzophenone-5-sulfonate sodium salt and phenylbenzimidazolesulfonic acid.

The amount of the aforementioned sun protection filters (one or more compounds) in the formulations of the invention is preferably from 0.001% to 30.0% by weight, particularly preferably from 0.05% to 20.0% by weight and especially preferably from 1.0% to 10.0% by weight, based on the total weight of the completed formulation.

The formulations of the invention can comprise one or more antioxidants, preferably selected from amino acids (e.g., glycine, histidine, tyrosine, tryptophan) and derivatives thereof, imidazoles (e.g., urocanic acid) and derivatives thereof, peptides such as DL-carnosine, D-carnosine, L-carnosine and derivatives thereof (e.g., anserine), carotenoids, carotenes (e.g., α-carotene, β-carotene, lycopene) and derivatives thereof, chlorogenic acid and derivatives thereof, lipoic acid and derivatives thereof (e.g., dihydrolipoic acid), aurothioglucose, propylthiouracil and other thiols (e.g., thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl, and glyceryl esters thereof), and also salts thereof, dilauryl thiodipropionate, distearyl thiodipropionate, thiodipropionic acid, and derivatives thereof (e.g., esters, ethers, peptides, lipids, nucleotides, nucleosides, and salts), and also sulfoximine compounds (e.g., buthionine sulfoximines, homocysteine sulfoximine, buthionine sulfones, penta-, hexa-, heptathionine sulfoximine) in very low tolerated doses, also (metal) chelating agents (e.g., α-hydroxyfatty acids, palmitic acid, phytic acid, lactoferrin), α-hydroxy acids (e.g., citric acid, lactic acid, malic acid), humic acid, bile acid, bile extracts, bilirubin, biliverdin, EDTA, EGTA, and derivatives thereof, unsaturated fatty acids and derivatives thereof (e.g., γ-linolenic acid, linoleic acid, oleic acid), folic acid and derivatives thereof, ubiquinone and ubiquinol and derivatives thereof, vitamin C and derivatives (e.g., ascorbyl palmitate, Mg ascorbyl phosphate, ascorbyl acetate), tocopherols and derivatives (e.g., vitamin E acetate), vitamin A and derivatives (vitamin A palmitate), and coniferyl benzoate of benzoin resin, rutinic acid and derivatives thereof, α-glycosylrutin, ferulic acid, furfurylideneglucitol, carnosine, butylhydroxytoluene, butylhydroxyanisole, nordihydroguaiacic acid, nordihydroguaiaretic acid, trihydroxybutyrophenone, uric acid and derivatives thereof, mannose and derivatives thereof, zinc and derivatives thereof (e.g., ZnO, ZnSO₄), selenium and derivatives thereof (e.g., selenomethionine), stilbenes and derivatives thereof (e.g., stilbene oxide, trans-stilbene oxide), superoxide dismutase, and the derivatives suitable according to the invention (salts, esters, ethers, sugars, nucleotides, nucleosides, peptides, and lipids) of these specified substances.

The antioxidants can protect the skin and the hair against oxidative stress. Preferred antioxidants here are vitamin E and derivatives thereof, and vitamin A and derivatives thereof.

The amount of the one or more antioxidants in the formulations of the invention is preferably from 0.001% to 30.0% by weight, particularly preferably from 0.05% to 20.0% by weight, and especially preferably from 1% to 10.0% by weight, based on the total weight of the formulation.

Furthermore, humectants selected from the sodium salt of 2-pyrrolidone-5-carboxylate (NaPCA), guanidine; glycolic acid and salts thereof, lactic acid and salts thereof, glucosamines and salts thereof, lactamide monoethanolamine, acetamide monoethanolamine, urea, hydroxy acids, panthenol and derivatives thereof, for example D-panthenol (R-2,4-dihydroxy-N-(3-hydroxypropyl)-3,3-dimethylbutamide), DL-panthenol, calcium pantothenate, panthetine, pantotheine, panthenyl ethyl ether, isopropyl palmitate, glycerol and/or sorbitol can be used, preferably in amounts of from 0.1 to 15% by weight and particularly preferably from 0.5% to 5.0% by weight, based on the completed formulations.

Additionally, the formulations of the invention can comprise organic solvents. In principle, suitable organic solvents are all mono- or polyhydric alcohols. Preference is given to using alcohols having 1 to 4 carbon atoms, such as ethanol, propanol, isopropanol, n-butanol, isobutanol, tert-butanol, glycerol, and mixtures of said alcohols. Further preferred alcohols are polyethylene glycols with a relative molecular mass below 2000. In particular, the use of polyethylene glycol with a relative molecular mass between 200 and 600 and in amounts up to 45.0% by weight and of polyethylene glycol with a relative molecular mass between 400 and 600 in amounts of from 5.0% to 25.0% by weight is preferred. Further suitable solvents are, for example, triacetin (glycerol triacetate) and 1-methoxy-2-propanol.

The formulations of the invention may comprise one or more substances selected from colorants, examples being dyes and/or pigments. The dyes and/or pigments present in the formulations of the invention, not only organic but also inorganic dyes and pigments, are selected from the corresponding positive list of the German cosmetics ordinance or from the EC list of cosmetic colorants.

| Chemical or other name | CIN | Color |
|---|---|---|
| Pigment Green | 10006 | green |
| Acid Green 1 | 10020 | green |
| 2,4-Dinitrohydroxynaphthalene-7-sulfonic acid | 10316 | yellow |
| Pigment Yellow 1 | 11680 | yellow |
| Pigment Yellow 3 | 11710 | yellow |
| Pigment Orange 1 | 11725 | orange |
| 2,4-Dihydroxyazobenzene | 11920 | orange |
| Solvent Red 3 | 12010 | red |
| 1-(2′-Chloro-4′-nitro-1′-phenylazo)-2- | 12085 | red |
| hydroxynaphthalene | | |
| Pigment Red 3 | 12120 | red |
| Cerise Red; Sudan Red; Fat Red G | 12150 | red |
| Pigment Red 112 | 12370 | red |
| Pigment Red 7 | 12420 | red |
| Pigment Brown 1 | 12480 | brown |
| 4-(2′-Methoxy-5′-sulfonic acid diethylamide-1′- | 12490 | red |
| phenylazo)-3-hydroxy-5″-chloro-2″,4″- | | |
| dimethoxy-2-naphthoic acid anilide | | |
| Disperse Yellow 16 | 12700 | yellow |
| 1-(4-Sulfo-1-phenylazo)-4-aminobenzenesulfonic acid | 13015 | yellow |
| 2,4-Dihydroxyazobenzene-4′-sulfonic acid | 14270 | orange |
| 2-(2,4-Dimethylphenylazo-5-sulfonic acid)-1- | 14700 | red |
| hydroxy-naphthalene-4-sulfonic acid | | |
| 2-(4-Sulfo-1-naphthylazo)-1-naphthol-4-sulfonic acid | 14720 | red |
| 2-(6-Sulfo-2,4-xylylazo)-1-naphthol-5-sulfonic acid | 14815 | red |
| 1-(4′-Sulfophenylazo)-2-hydroxynaphthalene | 15510 | orange |
| 1-(2-Sulfonic acid-4-chloro-5-carboxylic acid-1- | 15525 | red |
| phenylazo)-2-hydroxynaphthalene | | |
| 1-(3-Methylphenylazo-4-sulfonic acid)-2- | 15580 | red |
| hydroxynaphthalene | | |
| 1-(4′,(8′)-Sulfonic acid naphthylazo)-2- | 15620 | red |
| hydroxynaphthalene | | |
| 2-Hydroxy-1,2′-azonaphthalene-1′-sulfonic acid | 15630 | red |
| 3-Hydroxy-4-phenylazo-2-naphthylcarboxylic acid | 15800 | red |
| 1-(2-Sulfo-4-methyl-1-phenylazo)-2- | 15850 | red |
| naphthylcarboxylic acid | | |
| 1-(2-Sulfo-4-methyl-5-chloro-1-phenylazo)-2- | 15865 | red |
| hydroxy-naphthalene-3-carboxylic acid | | |
| 1-(2-Sulfo-1-naphthylazo)-2-hydroxynaphthalene- | 15880 | red |
| 3-carboxylic acid | | |
| 1-(3-Sulfo-1-phenylazo)-2-naphthol-6-sulfonic acid | 15980 | orange |
| 1-(4-Sulfo-1-phenylazo)-2-naphthol-6-sulfonic acid | 15985 | yellow |
| Allura Red | 16035 | red |
| 1-(4-Sulfo-1-naphthylazo)-2-naphthol-3,6- | 16185 | red |
| disulfonic acid | | |
| Acid Orange 10 | 16230 | orange |
| 1-(4-Sulfo-1-naphthylazo)-2-naphthol-6,8- | 16255 | red |
| disulfonic acid | | |
| 1-(4-Sulfo-1-naphthylazo)-2-naphthol-3,6,8- | 16290 | red |
| trisulfonic acid | | |
| 8-Amino-2-phenylazo-1-naphthol-3,6-disulfonic acid | 17200 | red |
| Acid Red 1 | 18050 | red |
| Acid Red 155 | 18130 | red |
| Acid Yellow 121 | 18690 | yellow |
| Acid Red 180 | 18736 | red |
| Acid Yellow 11 | 18820 | yellow |
| Acid Yellow 17 | 18965 | yellow |
| 4-(4-Sulfo-1-phenylazo)-1-(4-sulfophenyl)-5- | 19140 | yellow |
| hydroxypyrazolone-3-carboxylic acid | | |
| Pigment Yellow 16 | 20040 | yellow |
| 2,6-(4′-Sulfo-2″,4″-dimethyl)bisphenylazo)-1,3- | 20170 | orange |
| dihydroxybenzene | | |
| Acid Black 1 | 20470 | black |
| Pigment Yellow 13 | 21100 | yellow |
| Pigment Yellow 83 | 21108 | yellow |
| Solvent Yellow | 21230 | yellow |
| Acid Red 163 | 24790 | red |
| Acid Red 73 | 27290 | red |
| 2-[4′-(4″-Sulfo-1″-phenylazo)-7′-sulfo-1′- | 27755 | black |
| naphthylazo]-1-hydroxy-7-aminonaphthalene- | | |
| 3,6-disulfonic acid | | |
| 4′-[(4″-Sulfo-1″-phenylazo)-7′-sulfo-1′-naphthylazo]- | 28440 | black |
| 1-hydroxy-8-acetylaminonaphthalene-3,5- | | |
| disulfonic acid | | |
| Direct Orange 34, 39, 44, 46, 60 | 40215 | orange |
| Food Yellow | 40800 | orange |
| trans-β-Apo-8′-Carotenealdehyde (C₃₀) | 40820 | orange |
| trans-Apo-8′-Carotenic acid (C₃₀)-ethyl ester | 40825 | orange |
| Canthaxanthin | 40850 | orange |
| Acid Blue 1 | 42045 | blue |
| 2,4-Disulfo-5-hydroxy-4′-4″-bis(diethylamino) | 42051 | blue |
| triphenyl-carbinol | | |
| 4-[(4-N-Ethyl-p-sulfobenzylamino)phenyl-(4- | 42053 | green |
| hydroxy-2-sulfophenyl)(methylene)-1-(N-ethyl-N- | | |
| p-sulfobenzyl)-2,5-cyclohexadieneimine] | | |
| Acid Blue 7 | 42080 | blue |
| (N-Ethyl-p-sulfobenzylaminophenyl-(2- | 42090 | blue |
| sulfophenyl)-methylene-(N-ethyl-N-p- | | |
| sulfobenzyl)cyclohexadieneimine | | |
| Acid Green 9 | 42100 | green |
| Diethyldisulfobenzyldi-4-amino-2-chlorodi-2- | 42170 | green |
| methyl-fuchsonimmonium | | |
| Basic Violet 14 | 42510 | violet |
| Basic Violet 2 | 42520 | violet |
| 2′-Methyl-4′-(N-ethyl-N-m-sulfobenzyl)amino- | 42735 | blue |
| 4″-(N-diethyl)-amino-2-methyl-N-ethyl-N-m- | | |
| sulfobenzylfuchsonimmonium | | |
| 4′-(N-Dimethyl)amino-4″-(N-phenyl)aminonaphtho- | 44045 | blue |
| N-dimethylfuchsonimmonium | | |
| 2-Hydroxy-3,6-disulfo-4,4′- | 44090 | green |
| bisdimethylaminonaphtho-fuchsinimmonium | | |
| Acid Red | 45100 | red |
| 3-(2′-Methylphenylamino)-6-(2′-methyl-4′- | 45190 | violet |
| sulfophenylamino)-9-(2″-carboxyphenyl) | | |
| xanthenium salt | | |
| Acid Red 50 | 45220 | red |
| Phenyl-2-oxyfluorone-2-carboxylic acid | 45350 | yellow |
| 4,5-Dibromofluorescein | 45370 | orange |
| 2,4,5,7-Tetrabromofluorescein | 45380 | red |
| Solvent Dye | 45396 | orange |
| Acid Red 98 | 45405 | red |
| 3′,4′,5′,6′-Tetrachloro-2,4,5,7-tetrabromofluorescein | 45410 | red |
| 4,5-Diiodofluorescein | 45425 | red |
| 2,4,5,7-Tetraiodofluorescein | 45430 | red |
| Quinophthalone | 47000 | yellow |
| Quinophthalonedisulfonic acid | 47005 | yellow |
| Acid Violet 50 | 50325 | violet |
| Acid Black 2 | 50420 | black |
| Pigment Violet 23 | 51319 | violet |
| 1,2-Dioxyanthraquinone, calcium-aluminum complex | 58000 | red |
| 3-Oxypyrene-5,8,10-sulfonic acid | 59040 | green |
| 1-Hydroxy-4-N-phenylaminoanthraquinone | 60724 | violet |
| 1-Hydroxy-4-(4′-methylphenylamino)anthraquinone | 60725 | violet |
| Acid Violet 23 | 60730 | violet |
| 1,4-Di(4′-methylphenylamino)anthraquinone | 61565 | green |
| 1,4-Bis(o-sulfo-p-toluidine)anthraquinone | 61570 | green |
| Acid Blue 80 | 61585 | blue |
| Acid Blue 62 | 62045 | blue |
| N,N′-Dihydro-1,2,1′,2′-anthraquinoneazine | 69800 | blue |
| Vat Blue 6; Pigment Blue 64 | 69825 | blue |
| Vat Orange 7 | 71105 | orange |
| Indigo | 73000 | blue |
| Indigodisulfonic acid | 73015 | blue |
| 4,4′-Dimethyl-6,6′-dichlorothioindigo | 73360 | red |
| 5,5′-Dichloro-7,7′-dimethylthioindigo | 73385 | violet |
| Quinacridone Violet 19 | 73900 | violet |
| Pigment Red 122 | 73915 | red |
| Pigment Blue 16 | 74100 | blue |
| Phthalocyanine | 74160 | blue |
| Direct Blue 86 | 74180 | blue |
| Chlorinated phthalocyanines | 74260 | green |
| Natural Yellow 6, 19; Natural Red 1 | 75100 | yellow |
| Bixin, Nor-Bixin | 75120 | orange |
| Lycopene | 75125 | yellow |
| trans-alpha, beta- or gamma-Carotene | 75130 | orange |
| Keto- and/or hydroxyl derivatives of carotene | 75135 | yellow |
| Guanine or pearlescent agents | 75170 | white |
| 1,7-Bis(4-hydroxy-3-methoxyphenyl)-1,6- | 75300 | yellow |
| heptadiene-3,5-dione | | |
| Complex salt (Na, Al, Ca) of carminic acid | 75470 | red |
| Chlorophyll a and b; copper compounds of | 75810 | green |
| the chlorophylls and chlorophyllines | | |
| Aluminum | 77000 | white |
| Aluminum hydrate | 77002 | white |
| Water-containing aluminum silicates | 77004 | white |
| Ultramarine | 77007 | blue |
| Pigment Red 101 and 102 | 77015 | red |
| Barium sulfate | 77120 | white |
| Bismuth oxychloride and its mixtures with mica | 77163 | white |
| Calcium carbonate | 77220 | white |
| Calcium sulfate | 77231 | white |
| Carbon | 77266 | black |
| Pigment Black 9 | 77267 | black |
| Carbo medicinalis vegetabilis | 77268:1 | black |
| Chromium oxide | 77288 | green |
| Chromium oxide, hydrated | 77289 | green |
| Pigment Blue 28, Pigment Green 14 | 77346 | green |
| Pigment Metal 2 | 77400 | brown |
| Gold | 77480 | brown |
| Iron oxides and hydroxides | 77489 | orange |
| Iron oxides and hydroxides | 77491 | red |
| Hydrated iron oxide | 77492 | yellow |
| Iron oxide | 77499 | black |
| Mixtures of iron(II) and iron(III) hexacyanoferrate | 77510 | blue |
| Pigment White 18 | 77713 | white |
| Manganese ammonium diphosphate | 77742 | violet |
| Manganese phosphate; Mn₃(PO₄)₂*•7H₂O | 77745 | red |
| Silver | 77820 | white |
| Titanium dioxide and its mixtures with mica | 77891 | white |
| Zinc oxide | 77947 | white |
| 6,7-Dimethyl-9-(1′-D-ribityl)isoalloxazine, | | yellow |
| lactoflavin | | |
| Caramel | | brown |
| Capsanthin, Capsorubin | | orange |
| Betanine | | red |
| Benzopyrilium salts, anthocyanines | | red |
| Aluminum, zinc, magnesium and calcium stearate | | white |
| Bromothymol Blue | | blue |
| Bromocresol Green | | green |
| Acid Red 195 | | red |

Also advantageous are oil-soluble natural dyes, such as, for example, paprika extracts, β-carotene, and cochineal.

Pearlescent pigments are also advantageously used, e.g., pearl essence (guanine/hypoxanthine mixed crystals from fish scales) and mother-of-pearl (ground mussel shells), monocrystalline pearlescent pigments, such as, for example, bismuth oxychloride (BiOCl), layer-substrate pigments, e.g., mica/metal oxide, silver-white pearlescent pigments of TiO₂, interference pigments (TiO₂, varying layer thickness), color luster pigments (Fe₂O₃) and combination pigments (TiO₂/Fe₂O₃, TiO₂/Cr₂O₃, TiO₂/Prussian blue, TiO₂/carmine).

Within the context of the present invention, effect pigments are to be understood as meaning pigments which bring about particular optical effects as a result of their refractive properties. Effect pigments impart luster or glitter effects to the treated surface (skin, hair, mucosa) or are able to optically conceal skin unevennesses and skin wrinkles through diffuse light scattering. As a particular embodiment of the effect pigments, interference pigments are preferred. Particularly suitable effect pigments are, for example, mica particles which are coated with at least one metal oxide. Besides mica, a sheet silicate, silica gel and other SiO₂ modifications are also suitable as carriers. A metal oxide which is often used for the coating is, for example, titanium oxide, to which, if desired, iron oxide can be admixed. The reflection properties can be influenced via the size and the shape (e.g., spherical, ellipsoidal, flattened, planar, nonplanar) of the pigment particles and also via the thickness of the oxide coating. Other metal oxides too, e.g., bismuth oxychloride (BiOCl), and the oxides of, for example, titanium, in particular the TiO₂ modifications anatase and rutile, and of aluminum, tantalum, niobium, zirconium, and hafnium. With magnesium fluoride (MgF₂) and calcium fluoride (fluorspar, CaF₂), too, it is possible to produce effect pigments.

The effects can be controlled not only via the particle size but also via the particle size distribution of the pigment assembly. Suitable particle size distributions range, for example, from 2-50 μm, 5-25 μm, 5-40 μm, 5-60 μm, 5-95 μm, 5-100 μm, 10-60 μm, 10-100 μm, 10-125 μm, 20-100 μm, 20-150 μm, and <15 μm. A broader particle size distribution, for example of 20-150 μm, brings about glittering effects, whereas a narrower particle size distribution of <15 μm provides a uniform satin appearance.

The formulations of the invention comprise effect pigments preferably in amounts of from 0.1% to 20.0% by weight, more preferably from 0.5% to 10.0% by weight and especially preferably from 1% to 5.0% by weight, in each case based on the total weight of the formulation.

Preferably suitable as pearlizing component are fatty acid monoalkanolamides, fatty acid dialkanolamides, monoesters or diesters of alkylene glycols, in particular ethylene glycol and/or propylene glycol or oligomers thereof, with higher fatty acids, such as, for example, palmitic acid, stearic acid and behenic acid, monoesters or polyesters of glycerol with carboxylic acids, fatty acids and metal salts thereof, ketosulfones or mixtures of the specified compounds. Particular preference is given to ethylene glycol distearates and/or polyethylene glycol distearates having on average 3 glycol units.

If the formulations of the invention comprise pearlizing compounds, these are preferably present in the formulations of the invention in an amount of from 0.1% to 15.0% by weight and more preferably in an amount of from 1.0% to 10.0% by weight.

Fragrance and/or perfume oils which may be used are individual odorant compounds, examples being the synthetic products of the ester, ether, aldehyde, ketone, alcohol, and hydrocarbon types. Odorant compounds of the ester type are, for example, benzyl acetate, phenoxyethyl isobutyrate, p-tert-butylcyclohexyl acetate, linalyl acetate, dimethylbenzylcarbinyl acetate, phenylethyl acetate, linalyl benzoate, benzyl formate, ethylmethylphenyl glycinate, allyl cyclohexyl propionate, styrallyl propionate, and benzyl salicylate. The ethers include, for example, benzyl ethyl ethers; the aldehydes include, for example, the linear alkanals having 8 to 18 carbon atoms, citral, citronellal, citronellyloxyacetaldehyde, cyclamenaldehyde, hydroxycitronellal, lilial, and bourgeonal; the ketones include, for example, the ionones, alpha-isomethylionone, and methyl cedryl ketone; the alcohols include anethol, citronellol, eugenol, geraniol, linalool, phenylethyl alcohol, and terpineol; and the hydrocarbons include primarily the terpenes and balsams. Preference is given to using mixtures of different odorants which together produce a pleasing scent note.

Perfume oils can also comprise natural odorant mixtures, as are accessible from vegetable or animal sources, e.g., pine oil, citrus oil, jasmine oil, lily oil, rose oil or ylang-ylang oil. Essential oils of relatively low volatility, which in most cases are used as aromatic components, are also suitable as perfume oils, e.g., sage oil, chamomile oil, clove oil, melissa oil, mint oil, cinnamon leaf oil, linden blossom oil, juniperberry oil, vetiver oil, olibanum oil, galbanum oil, and ladanum oil.

Acids or alkalis used for adjusting the pH are preferably mineral acids, in particular HCl, inorganic bases, in particular NaOH or KOH, and organic acids, in particular citric acid.

The formulations have pH values of preferably 2 to 10, more preferably from 3 to 9, with particular preference from 4.5 to 8, and exceptionally preferably from 5.5 to 7.5.

The examples and applications below are intended to illustrate the invention, but without restricting it to them. All percentage figures are in percent by weight (% by weight), unless explicitly indicated otherwise.

### EXAMPLES

The listing below shows 285 copolymers of the invention that are suitable with particular advantage for formulating the compositions of the invention. The various copolymers #1 to #285 are obtainable in accordance with preparation processes a), b1), and b2) below.

### Process a):

These polymers are preparable by means of radical polymerization in tert-butanol. For this procedure, the monomers were introduced into tert-butanol, the reaction mixture was rendered inert, and then the reaction was started, after initial heating to 60° C., by addition of the corresponding tert-butanol-soluble initiator (preferably dilauroyl peroxide). After the end of reaction (two hours), the polymers are isolated by distillative removal of the solvent, followed by vacuum drying.

Copolymer # 1

| | | |
|---|---|---|
| 350 | g | tert-butanol with 2.5% water |
| 58.2 | g | stearyl acrylate |
| 1.8 | g | [2-(acryloyloxy)-ethyl]trimethylammonium chloride (AOETAC) |
| 2.0 | g | dilauroyl peroxide (DLP) |

### Procedure:

350 g of tert-butanol and 58.2 g of stearyl acrylate are charged to a 1 l flask with stirrer, temperature sensor, reflux condenser, and N₂ introduction facility, and the flask is closed. It is then rendered inert with N₂, after which 1.8 g of [2-(acryloyloxy)-ethyl]trimethylammonium chloride (AOETAC) are added. At an internal temperature of 60° C., the reaction is initiated with 2.0 g of dilauroyl peroxide (DLP). The resultant polymer remains in solution. The solution is subsequently stirred, after which the internal temperature is increased to reflux temperature, and subsequent stirring takes place to a sufficient degree. The product is dried in a vacuum drying oven at 35° C.

### Process b):

These polymers are preparable by the solution process in organic solvents (preferably toluene, also tertiary alcohols, for example). Here, the monomers are introduced into the solvent, the reaction mixture is rendered inert, and then the reaction is started, after heating to 60° C., by addition of suitable initiators or initiator systems (preferably dilauroyl peroxide). After the end of reaction, the polymers are precipitated by evaporation or cooling of the solvent or else isolated by precipitation from a solvent in which the polymer is insoluble, and are subsequently vacuum-dried.

Process b1): One-Pot Process

Copolymer # 276

| | |
|---|---|
| 350 g | tert-butanol with 2.5% water |
| 58.2 g | stearyl acrylate |
| 1.8 g | [2-(acryloyloxy)-ethyl]trimethylammonium |
| | chloride (AOETAC), and |
| 6.0 g | lauryl methacrylate |
| 2.0 g | dilauroyl peroxide (DLP) |

### Procedure:

350 g of tert-butanol and 58.2 g of stearyl acrylate are charged to a 1 l flask with stirrer, temperature sensor, reflux condenser, and N₂ introduction facility, and the flask is closed. It is then rendered inert with N₂, after which 1.8 g of [2-(acryloyloxy)-ethyl]trimethylammonium chloride (AOETAC) and 6.0 g of lauryl methacrylate are added. At an internal temperature of 60° C., the reaction is initiated with 2.0 g of dilauroyl peroxide (DLP). The polymer formed remains in solution. The solution is subsequently stirred and then the internal temperature is raised to reflux temperature. After a sufficient subsequent stirring period, the heating is switched off and the system is cooled to room temperature (RT). The polymer precipitates. The precipitate is isolated by suction filtration on a suction filter, and the product is washed and dried in a vacuum drying oven at 35° C.

Process b2): Precipitation from Cold Water

Copolymer # 1

| | |
|---|---|
| 350 g | tert-butanol with 2.5% water |
| 58.2 g | stearyl acrylate |
| 1.8 g | [2-(acryloyloxy)-ethyl]trimethylammonium chloride (AOETAC) |
| 2.0 g | dilauroyl peroxide (DLP) |

### Procedure:

350 g of tert-butanol and 58.2 g of stearyl acrylate are charged to a 1 l flask with stirrer, temperature sensor, reflux condenser, and N₂ introduction facility, and the flask is closed. It is then rendered inert with N₂, after which 1.8 g of [2-(acryloyloxy)-ethyl]trimethylammonium chloride (AOETAC) are added. At an internal temperature of 60° C., the reaction is initiated with 2.0 g of dilauroyl peroxide (DLP). The polymer formed remains in solution. The solution is subsequently stirred, after which the internal temperature is increased to reflux temperature, followed by subsequent stirring to a sufficient degree. The hot batch is then pumped, by means of a pump, into 700 g of ice-cold water. A white precipitate is formed. This precipitate is filtered off with suction, cold, on a white-ribbon circular filter, and dried in a vacuum drying oven at 35° C.

Table A below lists examples of polymers of the invention. In the first column of the table, monomers of the following structural formula (Ia1)

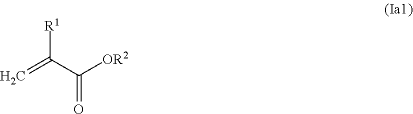

of component a) are shown.

TABLE A
Examples of polymers of the invention

| Polymer | | | | | | Preparation |
|---|---|---|---|---|---|---|
| # | Component a) | Component b) | Component c) | Component a) | Component a) or c) | route |
| | Below: | | | | | |
| | R² of the formula | | | | | |
| | (la1) with R¹ = H | | | | | |
| 1 | 97% C₁₈H₃₇ | 3% AOETAC | — | — | — | a |
| 2 | 97% C₁₈H₃₇ | 2% AOETAC | 1% EDDMA | — | — | a |
| 3 | 98% C₁₈H₃₇ | 1% AOETAC | 1% HDDMA | — | — | a |
| 4 | 97% C₁₈H₃₇ | 2% AOETAC | 1% HDDMA | — | — | a |
| 5 | 96% C₁₈H₃₇ | 3% AOETAC | 1% HDDMA | — | — | a |
| 6 | 94% C₁₈H₃₇ | 5% AOETAC | 1% HDDMA | — | — | a |
| 7 | 89% C₁₈H₃₇ | 10% AOETAC | 1% HDDMA | — | — | a |
| 8 | 96.5% C₁₈H₃₇ | 2% AOETAC | 1.5% HDDMA | — | — | a |
| 9 | 96% C₁₈H₃₇ | 2% AOETAC | 2% HDDMA | — | — | a |
| 10 | 95% C₁₈H₃₇ | 2% AOETAC | 3% HDDMA | — | — | a |
| 11 | 93% C₁₈H₃₇ | 2% AOETAC | 5% HDDMA | — | — | a |
| 12 | 97% C₁₈H₃₇ | 2% AOETAC | 1% BDDMA | — | — | a |
| 13 | 97% C₁₈H₃₇ | 2% AOETAC | 1% DDDMA | — | — | a |
| 14 | 97% C₁₈H₃₇ | 2% AOETAC | 1% TMPTA | — | — | a |
| 15 | 97% C₁₈H₃₇ | 2% AOETAC | 1% TMPTMA | — | — | a |
| 16 | 97% C₁₈H₃₇ | 2% AOETAC | 1% MBA | — | — | a |
| 17 | 97% C₁₈H₃₇ | 2% AOETAC | 1% HDDMA/TMPTA | — | — | a |
| 18 | 97% C₁₈H₃₇ | 2% AOETAC | 1% AMA | — | — | a |
| 19 | 97% C₁₈H₃₇ | 2% AOETAC | 1% Glycerol | — | — | a |
| | | | diacrylate | | | |
| 20 | 97% C₁₈H₃₇ | 2% AOETAC | 1% Triallylamine | — | — | a |
| 21 | 97% C₁₈H₃₇ | 2% AOETAC | 1% Triallylcyanurate | — | — | a |
| 22 | 97% C₁₈H₃₇ | 2% AOETAC | 1% PEG diacrylate | — | — | a |
| 23 | 93% C₁₈H₃₇ | 3% AOETAC | — | 4% B250MA | — | a |
| 24 | 89% C₁₈H₃₇ | 3% AOETAC | — | 8% B250MA | — | a |
| 25 | 87% C₁₈H₃₇ | 3% AOETAC | — | 10% B250MA | — | a |
| 26 | 85% C₁₈H₃₇ | 3% AOETAC | — | 12% B250MA | — | a |
| 27 | 91% C₁₈H₃₇ | 3% AOETAC | 1% HDDMA | 5% B250MA | — | a |
| 28 | 86% C₁₈H₃₇ | 3% AOETAC | 1% HDDMA | 10% B250MA | — | a |
| 29 | 76.2% C₁₈H₃₇ | 3% AOETAC | 1% HDDMA | 19.8% B250MA | — | a |
| 30 | 56.4% C₁₈H₃₇ | 3% AOETAC | 1% HDDMA | 39.6% B250MA | — | a |
| 31 | 82.9% C₁₈H₃₇ | 2.9% AOETAC | 4.7% HDDMA | 9.5% B250MA | — | a |
| 32 | 79.1% C₁₈H₃₇ | 2.7% AOETAC | 9.1% HDDMA | 9.1% B250MA | — | a |
| 33 | 78.7% C₁₈H₃₇ | 2.7% AOETAC | 4.8% HDDMA | 9% B250MA | 4.8% Hexadecyl acrylate | a |
| 34 | 74.6% C₁₈H₃₇ | 2.5% AOETAC | 4.8% HDDMA | 8.6% B250MA | 9.5% Hexadecyl acrylate | a |
| 35 | 66.3% C₁₈H₃₇ | 2.3% AOETAC | 4.8% HDDMA | 7.6% B250MA | 19% Hexadecyl acrylate | a |
| 36 | 78.7% C₁₈H₃₇ | 2.7% AOETAC | 4.8% HDDMA | 9% B250MA | 4.8% Octyldodecyl | a |
| | | | | | acrylate | |
| 37 | 74.6% C₁₈H₃₇ | 2.5% AOETAC | 4.8% HDDMA | 8.6% B250MA | 9.5% Octyldodecyl acrylate | a |
| 38 | 66.3% C₁₈H₃₇ | 2.3% AOETAC | 4.8% HDDMA | 7.6% B250MA | 19% Octyldodecyl acrylate | a |
| 39 | 78.7% C₁₈H₃₇ | 2.7% AOETAC | 4.8% HDDMA | 9% B250MA | 4.8% Isostearyl acrylate | a |
| 40 | 74.6% C₁₈H₃₇ | 2.5% AOETAC | 4.8% HDDMA | 8.6% B250MA | 9.5% Isostearyl acrylate | a |
| 41 | 66.3% C₁₈H₃₇ | 2.3% AOETAC | 4.8% HDDMA | 7.6% B250MA | 19% Isostearyl acrylate | a |
| 42 | 78.7% C₁₈H₃₇ | 2.7% AOETAC | 4.8% HDDMA | 9% B250MA | 4.8% C₂₆-C₂₈ α-olefin | a |
| 43 | 74.6% C₁₈H₃₇ | 2.5% AOETAC | 4.8% HDDMA | 8.6% B250MA | 9.5% C₂₆-C₂₈ α-olefin | a |
| 44 | 66.3% C₁₈H₃₇ | 2.3% AOETAC | 4.8% HDDMA | 7.6% B250MA | 19% C₂₆-C₂₈ α-olefin | a |
| 45 | 84.2% C₁₈H₃₇ | 1.6% AOETAC | 3.3% DDDMA | 9.3% B250MA | 1.6% C₂₆-C₂₈ α-olefin | a |
| 46 | 82.8% C₁₈H₃₇ | 4.2% AOETAC | 3.3% DDDMA | 4.7% B250MA | 5% C₂₆-C₂₈ α-olefin | a |
| 47 | 78.4% C₁₈H₃₇ | 5.2% AOETAC | 5% DDDMA | 4.7% B250MA | 6.7% C₂₆-C₂₈ α-olefin | a |
| 48 | 78.7% C₁₈H₃₇ | 2.7% AOETAC | 4.8% DDDMA | 9% B250MA | 4.8% C₃₀ α-olefin | a |
| 49 | 81.3% C₁₈H₃₇ | 2.8% AOETAC | 1.6% TMPTA | 9.3% B250MA | 5% C₂₆-C₂₈ α-olefin | a |
| 50 | 80% C₁₈H₃₇ | 2.8% AOETAC | 3.2% TMPTA | 9.2% B250MA | 4.8% C₂₆-C₂₈ α-olefin | a |
| 51 | 81.9% C₁₈H₃₇ | 1.6% AOETAC | 3.3% TMPTA | 9.9% B250MA | 3.3% C₂₆-C₂₈ α-olefin | a |
| 52 | 84.1% C₁₈H₃₇ | 1.6% AOETAC | 3.3% TMPTA | 9.4% B250MA | 1.6% C₂₆-C₂₈ α-olefin | a |
| 53 | 88.5% C₁₈H₃₇ | 1.7% AOETAC | 3.4% TMPTA | 4.7% B250MA | 1.7% C₂₆-C₂₈ α-olefin | a |
| 54 | 81.3% C₁₈H₃₇ | 1.7% AOETAC | 3.3% TMPTA | 12% B250MA | 1.7% C₂₆-C₂₈ α-olefin | a |
| 55 | 80.7% C₁₈H₃₇ | 0.8% AOETAC | 3.3% TMPTA | 10.2% B250MA | 5% C₂₆-C₂₈ α-olefin | a |
| 56 | 83.2% C₁₈H₃₇ | 1.7% AOETAC | 3.3% TMPTA | 8.4% B250MA | 3.4% C₂₆-C₂₈ α-olefin | a |
| 57 | 78.7% C₁₈H₃₇ | 2.7% AOETAC | 4.8% TMPTA | 9% B250MA | 4.8% C₂₆-C₂₈ α-olefin | a |
| 58 | 82.5% C₁₈H₃₇ | 0.8% AOETAC | 5% TMPTA | 8.4% B250MA | 3.3% C₂₆-C₂₈ α-olefin | a |
| 59 | 82.9% C₁₈H₃₇ | 2.9% AOETAC | 4.7% HDDMA/TMPTA | 9.5% B250MA | — | a |
| 60 | 78.2% C₁₈H₃₇ | 3.6% AOETAC | 4.7% HDDMA/TMPTA | 9% B250MA | 4.7% C₂₆-C₂₈ α-olefin | a |
| 61 | 91% C₁₈H₃₇ | 3% AOETAC | 1% HDDMA | 5% T250MA | — | a |
| 62 | 86.1% C₁₈H₃₇ | 3% AOETAC | 1% HDDMA | 9.9% T250MA | — | a |
| 63 | 91% C₁₈H₃₇ | 3% AOETAC | 1% HDDMA | 5% T080MA | — | a |
| 64 | 86.1% C₁₈H₃₇ | 3% AOETAC | 1% HDDMA | 9.9% T080MA | | |
| 65 | 91% C₁₈H₃₇ | 3% AOETAC | 1% HDDMA | 5% LA070MA | — | a |
| 66 | 86.1% C₁₈H₃₇ | 3% AOETAC | 1% HDDMA | 9.9% LA070MA | | |
| 67 | 91% C₁₈H₃₇ | 3% AOETAC | 1% HDDMA | — | 5% Butyl acrylate | a |
| 68 | 86.1% C₁₈H₃₇ | 3% AOETAC | 1% HDDMA | — | 9.9% Butyl acrylate | a |
| 69 | 86.1% C₁₈H₃₇ | 3% AOETAC | 1% HDDMA | — | 9.9% Hexadecyl acrylate | a |
| 70 | 86.1% C₁₈H₃₇ | 3% AOETAC | 1% HDDMA | — | 9.9% Octyldodecyl | a |
| | | | | | acrylate | |
| 71 | 86.1% C₁₈H₃₇ | 3% AOETAC | 1% HDDMA | — | 9.9% Isostearyl acrylate | a |
| 72 | 86.1% C₁₈H₃₇ | 3% AOETAC | 1% HDDMA | — | 9.9% C₂₆-C₂₈ α-olefin | a |
| 73 | 86.1% C₁₈H₃₇ | 3% AOETAC | 1% HDDMA | — | 9.9% C₃₀ α-olefin | a |
| 74 | 97% C₁₈H₃₇ | 3% MAPTAC | — | — | — | a |
| 75 | 97% C₁₈H₃₇ | 2% MAPTAC | 1% EDDMA | — | — | a |
| 76 | 97% C₁₈H₃₇ | 2% MAPTAC | 1% HDDMA | — | — | a |
| 77 | 97% C₁₈H₃₇ | 2% MAPTAC | 1% BDDMA | — | — | a |
| 78 | 97%C₁₈H₃₇ | 2% MAPTAC | 1% DDDMA | — | — | a |
| 79 | 97% C₁₈H₃₇ | 2% MAPTAC | 1% TMPTA | — | — | a |
| 80 | 97% C₁₈H₃₇ | 2% MAPTAC | 1% TMPTMA | — | — | a |
| 81 | 97% C₁₈H₃₇ | 2% MAPTAC | 1% MBA | — | -— | a |
| 82 | 97% C₁₈H₃₇ | 2% MAPTAC | 1% HDDMA/TMPTA | — | — | a |
| 83 | 97% C₁₈H₃₇ | 2% MAPTAC | 1% AMA | — | — | a |
| 84 | 97% C₁₈H₃₇ | 2% MAPTAC | 1% Glycerol | — | — | a |
| | | | diacrylate | | | |
| 85 | 97% C₁₈H₃₇ | 2% MAPTAC | 1% Triallylamine | — | — | a |
| 86 | 97% C₁₈H₃₇ | 2% MAPTAC | 1% Triallylcyanurate | — | — | a |
| 87 | 97% C₁₈H₃₇ | 2% MAPTAC | 1% PEG diacrylate | — | — | a |
| 88 | 87% C₁₈H₃₇ | 3% MAPTAC | — | 10% B250MA | — | a |
| 89 | 86% C₁₈H₃₇ | 3% MAPTAC | 1% HDDMA | 10% B250MA | — | a |
| 90 | 78.7% C₁₈H₃₇ | 2.7% MAPTAC | 4.8% HDDMA | 9% B250MA | 4.8% Butyl acrylate | a |
| 91 | 78.7% C₁₈H₃₇ | 2.7% MAPTAC | 4.8% HDDMA | 9% B250MA | 4.8% Hexadecyl acrylate | a |
| 92 | 78.7% C₁₈H₃₇ | 2.7% MAPTAC | 4.8% HDDMA | 9% B250MA | 4.8% Octyldodecyl | a |
| | | | | | acrylate | |
| 93 | 78.7% C₁₈H₃₇ | 2.7% MAPTAC | 4.8% HDDMA | 9% B250MA | 4.8% Isostearyl acrylate | a |
| 94 | 78.7% C₁₈H₃₇ | 2.7% MAPTAC | 4.8% HDDMA | 9% B250MA | 4.8% C₂₆-C₂₈ α-olefin | a |
| 95 | 78.7% C₁₈H₃₇ | 2.7% MAPTAC | 4.8% HDDMA | 9% B250MA | 4.8% C₃₀ α-olefin | a |
| 96 | 78.7% C₁₈H₃₇ | 2.7% MAPTAC | 4.8% DDDMA | 9% B250MA | 4.8% C₂₆-C₂₈ α-olefin | a |
| 97 | 78.7% C₁₈H₃₇ | 2.7% MAPTAC | 4.8% DDDMA | 9% B250MA | 4.8% C₃₀ α-olefin | a |
| 98 | 78.7% C₁₈H₃₇ | 2.7% MAPTAC | 4.8% TMPTA | 9% B250MA | 4.8% C₂₆-C₂₈ α-olefin | a |
| 99 | 78.7% C₁₈H₃₇ | 2.7% MAPTAC | 4.8% TMPTA | 9% B250MA | 4.8% C₃₀ α-olefin | a |
| 100 | 78.7% C₁₈H₃₇ | 3% MAPTAC | 1% HDDMA/TMPTA | 10% B250MA | — | a |
| 101 | 78.7% C₁₈H₃₇ | 2.7% MAPTAC | 4.8% HDDMA/TMPTA | 9% B250MA | 4.8% C₂₆-C₂₈ α-olefin | a |
| 102 | 86% C₁₈H₃₇ | 3% MAPTAC | 1% HDDMA | 10% T250MA | — | a |
| 103 | 86% C₁₈H₃₇ | 3% MAPTAC | 1% HDDMA | 10% T080MA | — | a |
| 104 | 86% C₁₈H₃₇ | 3% MAPTAC | 1% HDDMA | 10% LA070MA | — | a |
| 105 | 86.1% C₁₈H₃₇ | 3% MAPTAC | 1% HDDMA | — | 9.9% Butyl acrylate | a |
| 106 | 86.1% C₁₈H₃₇ | 3% MAPTAC | 1% HDDMA | — | 9.9% Hexadecyl acrylate | a |
| 107 | 86.1% C₁₈H₃₇ | 3% MAPTAC | 1% HDDMA | — | 9.9% Octyldodecy | a |
| | | | | | acrylate | |
| 108 | 86.1% C₁₈H₃₇ | 3% MAPTAC | 1% HDDMA | — | 9.9% Isostearyl acrylate | a |
| 109 | 86.1% C₁₈H₃₇ | 3% MAPTAC | 1% HDDMA | — | 9.9% C₂₆-C₂₈ α-olefin | a |
| 110 | 86.1% C₁₈H₃₇ | 3% MAPTAC | 1% HDDMA | — | 9.9% C₃₀ α-olefin | a |
| 111 | 78.2% C₁₈H₃₇ | 3.6% AOETAC/ | 4.7% HDDMA/TMPTA | 9% B250MA | 4.7% C₂₆-C₂₈ α-olefin | a |
| | | MAPTAC | | | | |
| 112 | 97% C₁₈H₃₇ | 3% DADMAC | — | — | — | a |
| 113 | 97% C₁₈H₃₇ | 2% DADMAC | 1% EDDMA | — | — | a |
| 114 | 97% C₁₈H₃₇ | 2% DADMAC | 1% HDDMA | — | — | a |
| 115 | 97% C₁₈H₃₇ | 2% DADMAC | 1% BDDMA | — | — | a |
| 116 | 97% C₁₈H₃₇ | 2% DADMAC | 1% DDDMA | — | — | a |
| 117 | 97% C₁₈H₃₇ | 2% DADMAC | 1% TMPTA | — | — | a |
| 118 | 86% C₁₈H₃₇ | 3% DADMAC | 1% HDDMA | 10% B250MA | — | a |
| 119 | 78.7% C₁₈H₃₇ | 2.7% DADMAC | 4.8% HDDMA | 9% B250MA | 4.8% C₂₆-C₂₈ α-olefin | a |
| 120 | 78.7% C₁₈H₃₇ | 2.7% DADMAC | 4.8% HDDMA | 9% B250MA | 4.8% C₃₀ α-olefin | a |
| | Below: | | | | | |
| | R² of the formula | | | | | |
| | (la1) with R¹ = | | | | | |
| | Methyl | | | | | |
| 121 | 97% C₁₂H₂₅ | 3% AOETAC | — | — | — | a |
| 122 | 98% C₁₂H₂₅ | 1% AOETAC | 1% HDDMA | — | — | a |
| 123 | 97% C₁₂H₂₅ | 2% AOETAC | 1% HDDMA | — | — | a |
| 124 | 96% C₁₂H₂₅ | 3% AOETAC | 1% HDDMA | — | — | a |
| 125 | 94% C₁₂H₂₅ | 5% AOETAC | 1% HDDMA | — | — | a |
| 126 | 89% C₁₂H₂₅ | 10% AOETAC | 1% HDDMA | — | — | a |
| 127 | 97% C₁₂H₂₅ | 2% AOETAC | 1% DDDMA | — | — | a |
| 128 | 97% C₁₂H₂₅ | 2% AOETAC | 1% TMPTA | — | — | a |
| 129 | 97% C₁₂H₂₅ | 2% AOETAC | 1% HDDMA/TMPTA | — | — | a |
| 130 | 93% C₁₂H₂₅ | 3% AOETAC | — | 4% B250MA | — | a |
| 131 | 89% C₁₂H₂₅ | 3% AOETAC | — | 8% B250MA | — | a |
| 132 | 87% C₁₂H₂₅ | 3% AOETAC | — | 10% B250MA | — | a |
| 133 | 91% C₁₂H₂₅ | 3% AOETAC | 1% HDDMA | 5% B250MA | — | a |
| 134 | 86% C₁₂H₂₅ | 3% AOETAC | 1% HDDMA | 10% B250MA | — | a |
| 135 | 82.9% C₁₂H₂₅ | 2.9% AOETAC | 4.7% HDDMA | 9.5% B250MA | — | a |
| 136 | 78.7% C₁₂H₂₅ | 2.7% AOETAC | 4.8% HDDMA | 9% B250MA | 4.8% Hexadecyl acrylate | a |
| 137 | 74.6% C₁₂H₂₅ | 2.5% AOETAC | 4.8% HDDMA | 8.6% B250MA | 9.5% Hexadecyl acrylate | a |
| 138 | 78.7% C₁₂H₂₅ | 2.7% AOETAC | 4.8% HDDMA | 9% B250MA | 4.8% Octyldodecyl | a |
| | | | | | acrylate | |
| 139 | 74.6% C₁₂H₂₅ | 2.5% AOETAC | 4.8% HDDMA | 8.6% B250MA | 9.5% Octyldodecy | a |
| | | | | | acrylate | |
| 140 | 78.7% C₁₂H₂₅ | 2.7% AOETAC | 4.8% HDDMA | 9% B250MA | 4.8% Isostearyl acrylate | a |
| 141 | 74.6% C₁₂H₂₅ | 2.5% AOETAC | 4.8% HDDMA | 8.6% B250MA | 9.5% Isostearyl acrylate | a |
| 142 | 78.7% C₁₂H₂₅ | 2.7% AOETAC | 4.8% HDDMA | 9% B250MA | 4.8% C₂₆-C₂₈ α-olefin | a |
| 143 | 74.6% C₁₂H₂₅ | 2.5% AOETAC | 4.8% HDDMA | 8.6% B250MA | 9.5% C₂₆-C₂₈ α-olefin | a |
| 144 | 78.7% C₁₂H₂₅ | 2.7% AOETAC | 4.8% DDDMA | 9% B250MA | 4.8% C₃₀ α-olefin | a |
| 145 | 80% C₁₂H₂₅ | 2.8% AOETAC | 3.2% TMPTA | 9% B250MA | 4.8% C₂₆-C₂₈ α-olefin | a |
| 146 | 82.9% C₁₂H₂₅ | 2.9% AOETAC | 4.7% HDDMA/TMPTA | 9.5% B250MA | — | a |
| 147 | 78.2% C₁₂H₂₅ | 3.6% AOETAC | 4.7% HDDMA/TMPTA | 9% B250MA | 4.7% C₂₆-C₂₈ α-olefin | a |
| 148 | 91% C₁₂H₂₅ | 3% AOETAC | 1% HDDMA | 5% T250MA | — | a |
| 149 | 86.1% C₁₂H₂₅ | 3% AOETAC | 1% HDDMA | 9.9% T250MA | — | A |
| 150 | 91% C₁₂H₂₅ | 3% AOETAC | 1% HDDMA | 5% T080MA | — | A |
| 151 | 86.1% C₁₂H₂₅ | 3% AOETAC | 1% HDDMA | 9.9% T080MA | | |
| 152 | 91% C₁₂H₂₅ | 3% AOETAC | 1% HDDMA | 5% LA070MA | — | A |
| 153 | 86.1% C₁₂H₂₅ | 3% AOETAC | 1% HDDMA | 9.9% LA070MA | | |
| 154 | 91% C₁₂H₂₅ | 3% AOETAC | 1% HDDMA | — | 5% Butyl acrylate | a |
| 155 | 86.1% C₁₂H₂₅ | 3% AOETAC | 1% HDDMA | — | 9.9% Butyl acrylate | a |
| 156 | 86.1% C₁₂H₂₅ | 3% AOETAC | 1% HDDMA | — | 9.9% Hexadecyl acrylate | a |
| 157 | 86.1% C₁₂H₂₅ | 3% AOETAC | 1% HDDMA | — | 9.9% Octyldodecy | a |
| | | | | | acrylate | |
| 158 | 86.1% C₁₂H₂₅ | 3% AOETAC | 1% HDDMA | — | 9.9% Isostearyl acrylate | a |
| 159 | 86.1% C₁₂H₂₅ | 3% AOETAC | 1% HDDMA | — | 9.9% C₂₆-C₂₈ α-olefin | a |
| 160 | 86.1% C₁₂H₂₅ | 3% AOETAC | 1% HDDMA | — | 9.9% C₃₀ α-olefin | a |
| 161 | 97% C₁₂H₂₅ | 3% MAPTAC | — | — | — | a |
| 162 | 97% C₁₂H₂₅ | 2% MAPTAC | 1% HDDMA | — | — | a |
| 163 | 97% C₁₂H₂₅ | 2% MAPTAC | 1% DDDMA | — | — | a |
| 164 | 97% C₁₂H₂₅ | 2% MAPTAC | 1% TMPTA | — | — | a |
| 165 | 97% C₁₂H₂₅ | 2% MAPTAC | 1% HDDMA/TMPTA | — | — | a |
| 166 | 87% C₁₂H₂₅ | 3% MAPTAC | — | 10% B250MA | — | a |
| 167 | 86% C₁₂H₂₅ | 3% MAPTAC | 1% HDDMA | 10% B250MA | — | a |
| 168 | 78.7% C₁₂H₂₅ | 2.7% MAPTAC | 4.8% HDDMA | 9% B250MA | 4.8% Butyl acrylate | a |
| 169 | 78.7% C₁₂H₂₅ | 2.7% MAPTAC | 4.8% HDDMA | 9% B250MA | 4.8% Hexadecyl acrylate | a |
| 170 | 78.7% C₁₂H₂₅ | 2.7% MAPTAC | 4.8% HDDMA | 9% B250MA | 4.8% Octyldodecy | a |
| | | | | | acrylate | |
| 171 | 78.7% C₁₂H₂₅ | 2.7% MAPTAC | 4.8% HDDMA | 9% B250MA | 4.8% Isostearyl acrylate | a |
| 172 | 78.7% C₁₂H₂₅ | 2.7% MAPTAC | 4.8% HDDMA | 9% B250MA | 4.8% C₂₆-C₂₈ α-olefin | a |
| 173 | 78.7% C₁₂H₂₅ | 2.7% MAPTAC | 4.8% HDDMA | 9% B250MA | 4.8% C₃₀ α-olefin | a |
| 174 | 78.7% C₁₂H₂₅ | 2.7% MAPTAC | 4.8% DDDMA | 9% B250MA | 4.8% C₂₆-C₂₈ α-olefin | a |
| 175 | 78.7% C₁₂H₂₅ | 2.7% MAPTAC | 4.8% DDDMA | 9% B250MA | 4.8% C₃₀ α-olefin | a |
| 176 | 78.7% C₁₂H₂₅ | 2.7% MAPTAC | 4.8% TMPTA | 9% B250MA | 4.8% C₂₆-C₂₈ α-olefin | a |
| 177 | 78.7% C₁₂H₂₅ | 2.7% MAPTAC | 4.8% TMPTA | 9% B250MA | 4.8% C₃₀ α-olefin | a |
| 178 | 78.7% C₁₂H₂₅ | 3% MAPTAC | 1% HDDMA/TMPTA | 10% B250MA | — | a |
| 179 | 78.7% C₁₂H₂₅ | 2.7% MAPTAC | 4.8% HDDMA/TMPTA | 9% B250MA | 4.8% C₂₆-C₂₈ α-olefin | a |
| 180 | 86% C₁₂H₂₅ | 3% MAPTAC | 1% HDDMA | 10% T250MA | — | a |
| 181 | 86% C₁₂H₂₅ | 3% MAPTAC | 1% HDDMA | 10% T080MA | — | a |
| 182 | 86% C₁₂H₂₅ | 3% MAPTAC | 1% HDDMA | 10% LA070MA | — | a |
| 183 | 86.1% C₁₂H₂₅ | 3% MAPTAC | 1% HDDMA | — | 9.9% Butyl acrylate | a |
| 184 | 86.1% C₁₂H₂₅ | 3% MAPTAC | 1% HDDMA | — | 9.9% Hexadecyl acrylate | a |
| 185 | 86.1% C₁₂H₂₅ | 3% MAPTAC | 1% HDDMA | — | 9.9% Octyldodecy | a |
| | | | | | acrylate | |
| 186 | 86.1% C₁₂H₂₅ | 3% MAPTAC | 1% HDDMA | — | 9.9% Isostearyl acrylate | a |
| 187 | 86.1% C₁₂H₂₅ | 3% MAPTAC | 1% HDDMA | — | 9.9% C₂₆-C₂₈ α-olefin | a |
| 188 | 86.1% C₁₂H₂₅ | 3% MAPTAC | 1% HDDMA | — | 9.9% C₃₀ α-olefin | a |
| 189 | 78.2% C₁₂H₂₅ | 3.6% AOETAC/ | 4.7% HDDMA/TMPTA | 9% B250MA | 4.7% C₂₆-C₂₈ α-olefin | a |
| | | MAPTAC | | | | |
| 190 | 97% C₁₂H₂₅ | 3% DADMAC | — | — | — | a |
| 191 | 97% C₁₂H₂₅ | 2% DADMAC | 1% HDDMA | — | — | a |
| 192 | 97% C₁₂H₂₅ | 2% DADMAC | 1% DDDMA | — | — | a |
| 193 | 97% C₁₂H₂₅ | 2% DADMAC | 1% TMPTA | — | — | a |
| 194 | 86% C₁₂H₂₅ | 3% DADMAC | 1% HDDMA | 10% B250MA | — | a |
| 195 | 78.7% C₁₂H₂₅ | 2.7% DADMAC | 4.8% HDDMA | 9% B250MA | 4.8% C₂₆-C₂₈ α-olefin | a |
| 196 | 78.7% C₁₂H₂₅ | 2.7% DADMAC | 4.8% HDDMA | 9% B250MA | 4.8% C₃₀ α-olefin | a |
| | Below: | | | | | |
| | R² of the formula | | | | | |
| | (la1) | | | | | |
| | with R¹ = H | | | | | |
| 197 | 97% C₂₂H₄₅ | 3% AOETAC | — | — | — | a |
| 198 | 98% C₂₂H₄₅ | 1% AOETAC | 1% HDDMA | — | — | a |
| 199 | 97% C₂₂H₄₅ | 2% AOETAC | 1% HDDMA | — | — | a |
| 200 | 96% C₂₂H₄₅ | 3% AOETAC | 1% HDDMA | — | — | a |
| 201 | 94% C₂₂H₄₅ | 5% AOETAC | 1% HDDMA | — | — | a |
| 202 | 89% C₂₂H₄₅ | 10 % AOETAC | 1% HDDMA | — | — | a |
| 203 | 97% C₂₂H₄₅ | 2% AOETAC | 1% DDDMA | — | — | a |
| 204 | 97% C₂₂H₄₅ | 2% AOETAC | 1% TMPTA | — | — | a |
| 205 | 97% C₂₂H₄₅ | 2% AOETAC | 1% HDDMA/TMPTA | — | — | a |
| 206 | 93% C₂₂H₄₅ | 3% AOETAC | — | 4% B250MA | — | a |
| 207 | 89% C₂₂H₄₅ | 3% AOETAC | — | 8% B250MA | — | a |
| 208 | 87% C₂₂H₄₅ | 3% AOETAC | — | 10% B250MA | — | a |
| 209 | 91% C₂₂H₄₅ | 3% AOETAC | 1% HDDMA | 5% B250MA | — | a |
| 210 | 86% C₂₂H₄₅ | 3% AOETAC | 1% HDDMA | 10% B250MA | — | a |
| 211 | 82.9% C₂₂H₄₅ | 2.9% AOETAC | 4.7% HDDMA | 9.5% B250MA | — | a |
| 212 | 78.7% C₂₂H₄₅ | 2.7% AOETAC | 4.8% HDDMA | 9% B250MA | 4.8% Hexadecyl acrylate | a |
| 213 | 74.6% C₂₂H₄₅ | 2.5% AOETAC | 4.8% HDDMA | 8.6% B250MA | 9.5% Hexadecyl acrylate | a |
| 214 | 78.7% C₂₂H₄₅ | 2.7% AOETAC | 4.8% HDDMA | 9% B250MA | 4.8% Octyldodecyl | a |
| | | | | | acrylate | |
| 215 | 74.6% C₂₂H₄₅ | 2.5% AOETAC | 4.8% HDDMA | 8.6 % B250MA | 9.5% Octyldodecyl | a |
| | | | | | acrylate | |
| 216 | 78.7% C₂₂H₄₅ | 2.7% AOETAC | 4.8% HDDMA | 9% B250MA | 4.8% Isostearyl acrylate | a |
| 217 | 74.6% C₂₂H₄₅ | 2.5% AOETAC | 4.8% HDDMA | 8.6% B250MA | 9.5% Isostearyl acrylate | a |
| 218 | 78.7% C₂₂H₄₅ | 2.7% AOETAC | 4.8% HDDMA | 9% B250MA | 4.8% C₂₆-C₂₈ α-olefin | a |
| 219 | 74.6% C₂₂H₄₅ | 2.5% AOETAC | 4.8% HDDMA | 8.6% B250MA | 9.5% C₂₆-C₂₈ α-olefin | a |
| 220 | 78.7% C₂₂H₄₅ | 2.7% AOETAC | 4.8% DDDMA | 9% B250MA | 4.8% C₃₀ α-olefin | a |
| 221 | 80% C₂₂H₄₅ | 2.8% AOETAC | 3.2% TMPTA | 9.2% B250MA | 4.8% C₂₆-C₂₈ α-olefin | a |
| 222 | 82.9% C₂₂H₄₅ | 2.9% AOETAC | 4.7% HDDMA/TMPTA | 9.5% B250MA | — | a |
| 223 | 78.2% C₂₂H₄₅ | 3.6% AOETAC | 4.7% HDDMA/TMPTA | 9% B250MA | 4.7% C₂₆-C₂₈ α-olefin | a |
| 224 | 91% C₂₂H₄₅ | 3% AOETAC | 1% HDDMA | 5% T250MA | — | a |
| 225 | 86.1% C₂₂H₄₅ | 3% AOETAC | 1% HDDMA | 9.9% T250MA | — | a |
| 226 | 91% C₂₂H₄₅ | 3% AOETAC | 1% HDDMA | 5% T080MA | — | a |
| 227 | 86.1% C₂₂H₄₅ | 3% AOETAC | 1% HDDMA | 9.9% T080MA | | |
| 228 | 91% C₂₂H₄₅ | 3% AOETAC | 1% HDDMA | 5% LA070MA | — | a |
| 229 | 86.1% C₂₂H₄₅ | 3% AOETAC | 1% HDDMA | 9.9% LA070MA | | |
| 230 | 91% C₂₂H₄₅ | 3% AOETAC | 1% HDDMA | — | 5% Butyl acrylate | a |
| 231 | 86.1% C₂₂H₄₅ | 3% AOETAC | 1% HDDMA | — | 9.9% Butyl acrylate | a |
| 232 | 86.1% C₂₂H₄₅ | 3% AOETAC | 1% HDDMA | — | 9.9% Hexadecyl acrylate | a |
| 233 | 86.1% C₂₂H₄₅ | 3% AOETAC | 1% HDDMA | — | 9.9% Octyldodecyl | a |
| | | | | | acrylate | |
| 234 | 86.1% C₂₂H₄₅ | 3% AOETAC | 1% HDDMA | — | 9.9% Isostearyl acrylate | a |
| 235 | 86.1% C₂₂H₄₅ | 3% AOETAC | 1% HDDMA | — | 9.9% C₂₆-C₂₈ α-olefin | a |
| 236 | 86.1% C₂₂H₄₅ | 3% AOETAC | 1% HDDMA | — | 9.9% C₃₀ α-olefin | a |
| 237 | 97% C₂₂H₄₅ | 3% MAPTAC | — | — | — | a |
| 238 | 97% C₂₂H₄₅ | 2% MAPTAC | 1% HDDMA | — | — | a |
| 239 | 97% C₂₂H₄₅ | 2% MAPTAC | 1% DDDMA | — | — | a |
| 240 | 97% C₂₂H₄₅ | 2% MAPTAC | 1% TMPTA | — | — | a |
| 241 | 97% C₂₂H₄₅ | 2% MAPTAC | 1% HDDMA/TMPTA | — | — | a |
| 242 | 87% C₂₂H₄₅ | 3% MAPTAC | — | 10% B250MA | — | a |
| 243 | 86% C₂₂H₄₅ | 3% MAPTAC | 1% HDDMA | 10% B250MA | — | a |
| 244 | 78.7% C₂₂H₄₅ | 2.7% MAPTAC | 4.8% HDDMA | 9% B250MA | 4.8% Butyl acrylate | a |
| 245 | 78.7% C₂₂H₄₅ | 2.7% MAPTAC | 4.8% HDDMA | 9% B250MA | 4.8% Hexadecyl acrylate | a |
| 246 | 78.7% C₂₂H₄₅ | 2.7% MAPTAC | 4.8% HDDMA | 9% B250MA | 4.8% Octyldodecyl | a |
| | | | | | acrylate | |
| 247 | 78.7% C₂₂H₄₅ | 2.7% MAPTAC | 4.8% HDDMA | 9% B250MA | 4.8% Isostearyl acrylate | a |
| 248 | 78.7% C₂₂H₄₅ | 2.7% MAPTAC | 4.8% HDDMA | 9% B250MA | 4.8% C₂₆-C₂₈ α-olefin | a |
| 249 | 78.7% C₂₂H₄₅ | 2.7% MAPTAC | 4.8% HDDMA | 9% B250MA | 4.8% C₃₀ α-olefin | a |
| 250 | 78.7% C₂₂H₄₅ | 2.7% MAPTAC | 4.8% DDDMA | 9% B250MA | 4.8% C₂₆-C₂₈ α-olefin | a |
| 251 | 78.7% C₂₂H₄₅ | 2.7% MAPTAC | 4.8% DDDMA | 9% B250MA | 4.8% C₃₀ α-olefin | a |
| 252 | 78.7% C₂₂H₄₅ | 2.7% MAPTAC | 4.8% TMPTA | 9% B250MA | 4.8% C₂₆-C₂₈ α-olefin | a |
| 253 | 78.7% C₂₂H₄₅ | 2.7% MAPTAC | 4.8% TMPTA | 9% B250MA | 4.8% C₃₀ α-olefin | a |
| 254 | 78.7% C₂₂H₄₅ | 3% MAPTAC | 1% HDDMA/TMPTA | 10% B250MA | — | a |
| 255 | 78.7% C₂₂H₄₅ | 2.7% MAPTAC | 4.8% HDDMA/TMPTA | 9% B250MA | 4.8% C₂₆-C₂₈ α-olefin | a |
| 256 | 78.7% C₂₂H₄₅ | 3% MAPTAC | 1% HDDMA | 10% T250MA | — | a |
| 257 | 78.7% C₂₂H₄₅ | 3% MAPTAC | 1% HDDMA | 10% T080MA | — | a |
| 258 | 78.7% C₂₂H₄₅ | 3% MAPTAC | 1% HDDMA | 10% LA070MA | — | a |
| 259 | 86.1% C₂₂H₄₅ | 3% MAPTAC | 1% HDDMA | — | 9.9% Butyl acrylate | a |
| 260 | 86.1% C₂₂H₄₅ | 3% MAPTAC | 1% HDDMA | — | 9.9% Hexadecyl acrylate | a |
| 261 | 86.1% C₂₂H₄₅ | 3% MAPTAC | 1% HDDMA | — | 9.9% Octyldodecyl | a |
| | | | | | acrylate | |
| 262 | 86.1% C₂₂H₄₅ | 3% MAPTAC | 1% HDDMA | — | 9.9% Isostearyl acrylate | a |
| 263 | 86.1% C₂₂H₄₅ | 3% MAPTAC | 1% HDDMA | — | 9.9% C₂₆-C₂₈ α-olefin | a |
| 264 | 86.1% C₂₂H₄₅ | 3% MAPTAC | 1% HDDMA | — | 9.9% C₃₀ α-olefin | a |
| 265 | 78.2% C₂₂H₄₅ | 3.6% AOETAC/ | 4.7% HDDMA/TMPTA | 9% B250MA | 4.7% C₂₆-C₂₈ α-olefin | a |
| | | MAPTAC | | | | |
| 266 | 97% C₂₂H₄₅ | 3% DADMAC | — | — | — | a |
| 267 | 97% C₂₂H₄₅ | 2% DADMAC | 1% HDDMA | — | — | a |
| 268 | 97% C₂₂H₄₅ | 2% DADMAC | 1% DDDMA | — | — | a |
| 269 | 97% C₂₂H₄₅ | 2% DADMAC | 1% TMPTA | — | — | a |
| 270 | 86% C₂₂H₄₅ | 3% DADMAC | 1% HDDMA | 10% B250MA | — | a |
| 271 | 78.7% C₂₂H₄₅ | 2.7% DADMAC | 4.8% HDDMA | 9% B250MA | 4.8% C₂₆-C₂₈ α-olefin | a |
| 272 | 78.7% C₂₂H₄₅ | 2.7% DADMAC | 4.8% HDDMA | 9% B250MA | 4.8% C₃₀ α-olefin | a |
| 273 | 97% C₁₈H₃₇ | 3% AOETAC | — | — | — | b1 |
| 274 | 92.4% C₁₈H₃₇ | 2.8% AOETAC | 4.8% HDDMA | — | — | b1 |
| 275 | 92% C₁₈H₃₇ | 3% AOETAC | — | — | 5% Lauryl methacrylate | b1 |
| 276 | 87% C₁₈H₃₇ | 3% AOETAC | — | — | 10% Lauryl methacrylate | b1 |
| 277 | 82% C₁₈H₃₇ | 3% AOETAC | — | — | 15% Lauryl methacrylate | b1 |
| 278 | 97% C₁₈H₃₇ | 3% AOETAC | — | — | — | b2 |
| 279 | 92.4% C₁₈H₃₇ | 2.8% AOETAC | 4.8% HDDMA | — | — | b2 |
| 280 | 86% C₁₈H₃₇ | 3% AOETAC | 1% HDDMA | 10% B250MA | — | b2 |
| 281 | 92% C₁₈H₃₇ | 3% AOETAC | — | 5% B250MA | — | b2 |
| 282 | 87% C₁₈H₃₇ | 3% AOETAC | — | 10% B250MA | — | b2 |
| 283 | 82% C₁₈H₃₇ | 3% AOETAC | — | 15% B250MA | — | b2 |
| 284 | 77% C₁₈H₃₇ | 3% AOETAC | — | 20% B250MA | — | b2 |
| 285 | 78.7% C₁₈H₃₇ | 2.7% AOETAC | 4.8% HDDMA | 9% B250MA | 4.8% C₂₆-C₂₈ α-olefin | b2 |

Cosmetic formulations below were prepared with copolymers of the invention:

### Example 1

### W/O Cream

| | | |
|---|---|---|
| A | Liquid paraffin | 12.50% |
| | Isopropyl palmitate | 7.50% |
| | Soybean oil | 5.00% |
| | Magnesium stearate | 1.00% |
| | Copolymer #1 | 2.00% |
| B | Water | ad 100% |
| C | Preservative | q.s. |

### Preparation

I Melt components A at 80° C.
II Heat B to 80° C.
III Stir II into I
IV Add C at 35° C.

### Example 2

### W/O Cream

| | | |
|---|---|---|
| A | Decyl oleate | 7.50% |
| | Squalane | 7.50% |
| | Velsan ® CCT | 7.50% |
| | Caprylic-Capric triglyceride | |
| | Isopropyl palmitate | 7.50% |
| | Magnesium stearate | 0.67% |
| | Aluminum stearate | 0.33% |
| | Copolymer #1 | 2.50% |
| B | Water | ad 100% |
| | 1,2-propylene glycol | 2.00% |
| | Magnesium sulfate | 1.00% |
| C | Preservative | q.s. |
| | Tocopherol acetate | 0.10% |

### Preparation

I Melt components A at 80° C.
II Mix components B and heat to 80° C.
III Stir II into I
IV Add C at 35° C.

### Example 3

### W/O Cream

| | | |
|---|---|---|
| A | Polyglyceryl-2 sesquiisostearate | 2.00% |
| | Liquid paraffin | 12.50% |
| | Isopropyl palmitate | 7.50% |
| | Soybean oil | 5.00% |
| | Magnesium stearate | 1.00% |
| | Copolymer #1 | 2.00% |
| B | Water | ad 100% |
| C | Preservative | q.s. |

### Preparation

I Melt components A at 80° C.
II Heat component B to 80° C.
III Stir II into I
IV Add C at 35° C.

### Example 4

### W/O Cream

| | | |
|---|---|---|
| A | Polyglyceryl-2 sesquiisostearate | 2.00% |
| | Beeswax | 1.00% |
| | Microcrystalline wax | 1.00% |
| | Liquid paraffin | 12.50% |
| | Isopropyl palmitate | 7.50% |
| | Soybean oil | 5.00% |
| | Magnesium stearate | 1.00% |
| | Copolymer #1 | 1.00% |
| B | Water | ad 100 |
| C | Preservative | q.s. |

### Preparation

I Melt components A at 80° C.
II Heat component B to 80° C.
III Stir II into I
IV Add C at 35° C.

### Example 5

### W/O Cream

| | | |
|---|---|---|
| A | Beeswax | 2.00% |
| | Microcrystalline wax | 3.00% |
| | Liquid paraffin | 12.50% |
| | Isopropyl palmitate | 7.50% |
| | Soybean oil | 5.00% |
| | Magnesium stearate | 1.00% |
| | Copolymer #1 | 1.00% |
| B | Water | ad 100 |
| C | Preservative | q.s. |

### Preparation

I Melt components A at 80° C.
II Heat component B to 80° C.
III Stir II into I
IV Add C at 35° C.

### Example 6

### Cream Rinse

| | | |
|---|---|---|
| A | Hostacerin ® T3 | 1.50% |
| | Ceteareth-3 | |
| | Cetyl alcohol | 3.00% |
| | Copolymer #30 | 1.00% |
| | Genamin ® CTAC | 2.30% |
| | Cetrimonium Chloride | |
| B | Water | ad 100% |
| C | Citric acid | q.s. |

### Preparation

I Melt components A at 80° C.
II Add C to B to a pH of 3.7, and heat to 80° C.
III Stir II into I, continue stirring until cooling to 35° C.

### Example 7

### Cream Rinse

| | | |
|---|---|---|
| A | Hostacerin ® T3 | 1.50% |
| | Ceteareth-3 | |
| | Cetyl alcohol | 3.00% |
| | Copolymer #30 | 0.50% |
| | Genamin ® CTAC | 1.65% |
| | Cetrimonium chloride | |
| B | Water | ad 100% |
| C | Citric acid | q.s. |

### Preparation

I Melt components A at 80° C.
II Add C to B to a pH of 3.7
III Heat II to 80° C.
IV Stir III into I, continue stirring until cooling to 35° C.

### Example 8

### Sun Protection Cream Gel

| | | |
|---|---|---|
| A | Tegosoft ® TN | 8.00% |
| | C₁₂₋₁₅ alkyl benzoate | |
| | Velsan ® CCT | 5.00% |
| | Capryl-capric triglyceride | |
| | Eusolex ® OCR | 9.00% |
| | Octocrylene | |
| | Eusolex ® 2292 | 7.00% |
| | Ethylhexyl methoxycinnamate | |
| | Eusolex ® 9020 | 2.50% |
| | Butyl methoxydibenzoylmethane | |
| | Cetearyl alcohol | 1.00% |
| | Emulsogen ® HCO 040 | 1.00% |
| | PEG-40 hydrogenated castor oil | |
| | Copolymer # 1 | 2.00% |
| | Phenonip ® | q.s. |
| | Phenoxyethanol, methylparaben, | |
| | ethylparaben, butylparaben, | |
| | propylparaben, isobutylparaben | |
| B | Water | ad 100.00% |

### Preparation

I Melt components A at 70° C.
II Heat component B to 70° C.
III Stir II into I

### Example 9

### Cream Rinse

| | | |
|---|---|---|
| A | Dow Corning ® 5200 | 1.00% |
| | Laurylmethicone copolyol | |
| | Cetyl ester | 1.00% |
| | Cetearyl alcohol | 3.00% |
| B | Genamin ® KDMP | 0.50% |
| | Behentrimonium chloride | |
| | Copolymer # 30 | 1.00% |
| C | Water | ad 100.00% |
| | Preservative | q.s. |
| | Hydrotriticum WQ | 1.00% |
| D | Fragrance | 0.30% |
| | Dye | q.s. |
| | Dow Corning ® 949 | 2.00% |
| | Amodimethicone cetrimonium | |
| | chloride trideceth-12 | |

### Preparation

I Melt components A at 75° C.
II Stir B into C and heat to 75° C.
III Stir II into I, continue stirring until cooling to 30° C.
IV Add D at a temperature of 30° C. to III
V Adjust IV to pH 4.0

### Example 10

### Cream Rinse

| | | |
|---|---|---|
| A | Hostacerin ® DGI | 1.50% |
| | Polyglyceryl-2 sesquiisostearate | |
| | Cetyl alcohol | 4.00% |
| B | Genamin ® BTLF | 2.30% |
| | Behentrimonium chloride | |
| | Copolymer #30 | 1.00% |
| | Water | ad 100% |
| C | Fragrance | 0.30% |
| | Preservative | q.s. |
| | Dye | q.s. |
| | SilCare ® Silicone SEA | 1.00% |
| | Trideceth-9 PG-Amodimethicone | |
| | Trideceth-12 | |

### Preparation

I Melt components A at 70° C.
II Heat components B to 70° C.
III Stir II into I, continue stirring until cooling to 30° C.
IV Add C at 30° C. to III
V Adjust IV to pH 4.0

### Example 11

### Child Sun Protection

| | | |
|---|---|---|
| A | Abil ® EM 90 | 2.00% |
| | Cetyl PEG/PPG-10/1 dimethicone | |
| | Dow Corning ® 246 | 11.00% |
| | Cyclopentasiloxane/ | |
| | Cyclohexasiloxane | |
| | Titanium dioxide UV Titan M 262 | 10.00% |
| | Titanium dioxide/Aluminum oxide/ | |
| | Stearic acid | |
| | Crodamol AB | 12.00% |
| | C12-15 alkylbenzoate | |
| | Zinc oxide HP1 | 4.00% |
| | Zinc oxide | |
| | 1,3-butanediol | 3.00% |
| | Butylene glycol | |
| | Abil ® WE09 | 6.00% |
| | Hexyl laurate/ | |
| | Polyglyceryl-4 isostearate/ | |
| | cetyldimethicone | |
| | Copolymer #1 | 0.50% |
| | Aluminum/Magnesium hydroxide | 0.50% |
| | stearate | |
| | Aluminum stearate | 0.50% |
| | Cutina ® HR | 0.50% |
| | hydrogenated castor oil | |
| B | Water | ad 100% |
| | Glycerol | 5.00% |
| | Ginkgo Biloba extract (liquid) | 0.70% |
| | Sodium carboxymethyl betaglutan | 0.20% |
| | Polyglucose | |
| | Aquamollin BC powder Hoko | 0.20% |
| | Disodium EDTA | |
| C | Phenonip ® ME | 0.80% |
| | Methyl-/propylparaben/ | |
| | phenoxyethanol | |
| | Sodium chloride | 1.00% |
| | Aluminum hydroxide | 0.30% |

### Preparation

I Melt components A at 80° C.
II Heat components B to 80° C.
III Stir II into I
IV Add C to III after cooling to 35° C.

### Example 12

### Liquid Paraffin Gel

| | |
|---|---|
| Copolymer #277 | 5.00% |
| Liquid paraffin | 95.00% |

### Preparation

I Mix and heat the components until a homogeneous solution is obtained

The gel was assessed after cooling to room temperature. The resulting oil gel was colorless and transparent to slightly opaque.

### Example 13

### Thickening of Velsan® CCT (Capric/Caprylic Triglyceride, Clariant)

| | |
|---|---|
| Copolymer #60 | 5.00% |
| Velsan ® CCT | 95.00% |
| Caprylic/capric triglyceride | |

### Preparation

I Mix and heat the components until a homogeneous solution is obtained

The gel was assessed after cooling to room temperature. The resulting oil gel was amber and transparent to slightly opaque.

### Example 14

### Thickening of Rapeseed Oil Methyl Ester

| | |
|---|---|
| Copolymer #40 | 5.00% |
| Rapeseed oil methyl ester | 95.00% |

### Preparation

I Mix and heat the components until a homogeneous solution is obtained

The gel was assessed after cooling to room temperature. The resulting oil gel was deep yellow and transparent to slightly opaque.

### Example 15

### Thickening of a Sunflower Oil/Soya Oil (23:77% by Weight) Mixture

| | |
|---|---|
| Copolymer #52 | 5.00% |
| Sunflower oil/Soya oil | 95.00% |

### Preparation

I Mix and heat the components until a homogeneous solution is obtained

The gel was assessed after cooling to room temperature. The resulting oil gel was yellow and transparent to slightly opaque.

### Example A

Formulation examples 1-5, 8, and 11 were repeated, but replacing polymer #1 in each case by polymers #4, 25, 30, 31, 33, 39, 40, 44, 52, 60, 111, 273, 275, 276, 277, 278, and 282 from Table A in equal quantity. These formulations were stored at 40° C. and held at this temperature for six weeks. Stable formulations were obtained.

### Example B

Formulation examples 6, 7, 9, and 10 were repeated, replacing polymer #30 by polymers #1, 4, 25, 31, 33, 39, 40, 44, 52, 60, 111, 273, 275, 276, 277, 278, and 282 from Table A in equal quantity. Homogeneous cream rinses were obtained.

### Example C

In formulation examples 12-15, alternatively, 2% by weight of the corresponding polymers and 98% by weight of oil were used. Even with the reduced polymer fraction, good to very good thickening properties were achieved.

### Example D

Formulation examples 12-15 were repeated, using in formulation example 12, instead of polymer #277, the polymers #1, 4, 25, 30, 31, 33, 39, 40, 44, 52, 60, 111, 273, 275, 276, 278, and 282; in formulation example 13, instead of polymer #60, polymers #1, 4, 25, 30, 31, 33, 39, 40, 44, 52, 111, 273, 275, 276, 277, 278, and 282, in formulation example 14, instead of polymer #40, polymers #1, 4, 25, 30, 31, 33, 39, 44, 52, 60, 111, 273, 275, 276, 277, 278, and 282, in formulation example 15, instead of polymer #52, polymers #1, 4, 25, 30, 31, 33, 39, 40, 44, 60, 111, 273, 275, 276, 277, 278, and 282, from Table A, in equal quantity. The oil gels were assessed visually at 20° C. as being clear and transparent to slightly opaque.

### Example E

The formulation examples from example D were repeated, but using, instead of 5% by weight of the corresponding polymer, in each case 2% by weight of polymer and 98% by weight of oil. Even with the reduced polymer fraction, good to very good thickening properties were achieved.

### Example F

The formulation examples from example A-E were repeated, using, instead of the polymers used in examples A-E, the polymers #121, 123, 132, 135, 136, 140, 141, 145, 147, 189, 197, 199, 208, 211, 212, 216, 217, 221, 223, and 265 from Table A, in equal quantity.

Chemical or INCI designation of the trade names or abbreviations used
AMA Allyl methacrylate
AOETAC 2-(Acryloyloxy)ethyl]trimethylammonium chloride
APTAC [3-Acrylamidopropyl]trimethylammonium chloride
B250MA Behenyl methacrylate, ethoxylated with 25 EO units
BDDMA Buanediol dimethacrylate
DADMAC Diallyldimethylammonium chloride
DDDMA Dodecanediol dimethacrylate
EDDMA Ethanediol dimethacrylate
HDDMA Hexanediol dimethacrylate
HEMA 2-Hydroxyethyl methacrylate
LA070MA Lauryl methacrylate, ethoxylated with 7 EO units
MAOETAC [2-(Methacryloyloxy)ethyl]trimethylammonium chloride
MAPTAC [3-Methacrylamidopropyl]trimethylammonium chloride
MBA Methylene-bis-acrylamide
PEG diacrylate Polyethylene glycol diacrylate having an average
molecular weight Mₙ=575
Poly-NVP Poly-N-vinylpyrrolidone
T080MA Talyl methacrylate, ethoxylated with 8 EO units
T250MA Talyl methacrylate, ethoxylated with 25 EO units
TEMPO 2,2,6,6-Tetramethylpiperidinyloxyl
TMPTA Trimethylolpropane triacrylate
TMPTMA Trimethylolpropane trimethacrylate

1. A cosmetic, pharmaceutical or dermatological formulation comprising
a copolymer comprising
a) 60.0%-99.9% by weight of at least one structural unit originating from at least one polymerizable compound selected from group consisting of the structural formulae (Ia) and (Ib) 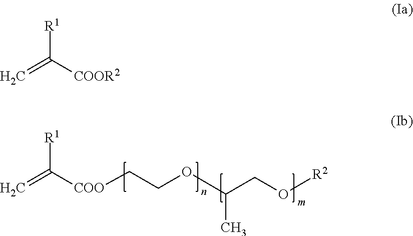
wherein
R¹ is hydrogen or methyl,
R² is a linear or branched saturated alkyl radical, having 12 to 200 carbon atoms, or a linear or branched mono- or polyunsaturated alkenyl radical having 12 to 200 carbon atoms,
n and m each independently of one another are an integer from 0 to 200 and the sum of n+m is a number from 1 to 200,
b) 0.1%-20.0% by weight of at least one structural unit, originating from at least one polymerizable quaternary ammonium compound, and
c) 0%-20.0% by weight of at least one nonionic structural unit originating from at least one further polymerizable substance,
and at least one oil,
with the proviso that the structural units a), b) and c) are not anionic, wherein the copolymer comprises no structural units originating from anionic polymerizable substances and wherein the copolymer thickens the oil.

2. (canceled)

3. (canceled)

4. The cosmetic, pharmaceutical or dermatological formulation as claimed in claim 1, wherein the at least one compound of the formula (Ia) is selected from the group consisting of lauryl acrylate, lauryl methacrylate, hexadecyl acrylate, hexadecyl methacrylate, stearyl acrylate, stearyl methacrylate, isostearyl acrylate, isostearyl methacrylate, octyldodecyl acrylate, octyldodecyl methacrylate, behenyl acrylate and behenyl methacrylate, and the at least one compound of the formula (Ib) is selected from the group consisting of acrylic ester of lauryl alcohol with 7 EO units, methacrylic ester of lauryl alcohol with 7 EO units, acrylic ester of talyl alcohol with 8 EO units, methacrylic ester of talyl alcohol with 8 EO units, acrylic ester of talyl alcohol with 25 EO units, methacrylic ester of talyl alcohol with 25 EO units, acrylic ester of behenyl alcohol with 25 EO units, and methacrylic ester of behenyl alcohol with 25 EO units.

5. The cosmetic, pharmaceutical or dermatological formulation as claimed in claim 1, wherein R² in the at least one compound of the formulae (Ia) and (Ib) is a linear or branched, saturated alkyl radical having 12 to 200 carbon atoms.

6. The cosmetic, pharmaceutical or dermatological formulation as claimed in claim 1, wherein the at least one structural unit of component b) originates from at least one substance selected from the group consisting of
b1) at least one polymerizable quaternary ammonium compound of the following structural formula (II)
X⁻⁺NR⁷R⁸R⁹R₁₀ (II)
wherein
X⁻ is a negatively charged counterion,
R⁷ is vinyl radical, allyl radical or a radical of the following structural formula (III) 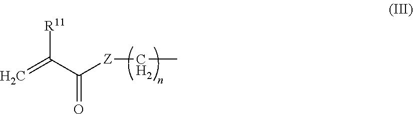
wherein
R¹¹ is hydrogen or methyl,
Z is O, NH, NCH₃ or S,
n is an integer from 1 to 50,
R⁸ is a linear or branched alkyl radical having 1 to 50 carbon atoms or is a radical R⁷,
R⁹ is a linear or branched alkyl radical having 1 to 50 carbon atoms or is a radical R⁷, and
R¹⁰ is a linear or branched alkyl radical having 1 to 50 carbon atoms, and
b2) at least one polymerizable cyclic quaternary ammonium compound of formula (IV) 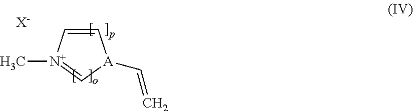
wherein
o and p each independently of one another are an integer from 0 to 5,
A is C, CH, N or P, and
X⁻ is a negatively charged counterion.

7. The cosmetic, pharmaceutical or dermatological formulation as claimed in claim 6, wherein the negatively charged counterion X⁻ in formula (II) and (IV) is selected from the group consisting of chloride, bromide, iodide, ½ SO₄²⁻ and methosulfate.

8. The cosmetic, pharmaceutical or dermatological formulation as claimed in claim 1, wherein the at least one structural unit of component b) originates from at least one polymerizable quaternary ammonium compound selected from the group consisting of diallyldimethylammonium chloride (DADMAC), [2-(methacryloyloxy)ethyl]trimethylammonium chloride (MAOETAC), [2-(acryloyloxy)ethyl]trimethylammonium chloride (AOETAC), [2-methacrylamidoethyl]trimethylammonium chloride, [2-(acrylamido)ethyl]trimethylammonium chloride, [3-methacrylamidopropyl]trimethylammonium chloride (MAPTAC), [3-acrylamidopropyl]trimethylammonium chloride (APTAC), N-methyl-2-vinylpyridinium chloride, N-methyl-4-vinylpyridinium chloride, and N-methyl-3-vinylimidazolinium chloride.

9. The cosmetic, pharmaceutical or dermatological formulation as claimed in claim 6, wherein the at least one structural unit of component b) originates from at least one polymerizable quaternary ammonium compound of formula (II).

10. The cosmetic, pharmaceutical or dermatological formulation as claimed in claim 1, comprising at least one structural unit of component c) that originates from substances selected from the group consisting of monomers having more than one polymerizable group, which may have a crosslinking effect.

11. The cosmetic, pharmaceutical or dermatological formulation as claimed in claim 1, wherein the at least one structural unit of component c) originates from substances selected from the group consisting of divinylbenzene, methylenebisacrylamide (MBA), triallylamine, triallyl cyanurate, ethanediol diacrylate, ethanediol dimethacrylate (EDDMA), buanediol diacrylate, buanediol dimethacrylate (BDDMA), hexanediol diacrylate, hexanediol dimethacrylate (HDDMA), dodecanediol diacrylate, dodecanediol dimethacrylate (DDDMA), trimethylolpropane triacrylate (TMPTA), trimethylolpropane trimethacrylate (TMPTMA), glycerol diacrylate, glycerol dimethacrylate, tetraethylene glycol diacrylate, tetraethylene glycol dimethacrylate, polyethylene glycol diacrylate with molar masses between 400 and 800 g/mol, polyethylene glycol dimethacrylate with molar masses between 400 and 800 g/mol, allyl acrylate, methacrylate or ethacrylate, preferably allyl acrylate or methacrylate, and more particularly allyl methacrylate (AMA), dipropylene glycol diallyl ether, polyglycol diallyl ether, hydroquinone diallyl ether, trimethylolpropane diallyl ether, trimethylolpropane triallyl ether, tetraallyloxyethane, triethylene glycol divinyl ether, other allyl ethers or vinyl ethers of polyfunctional alcohols, and mixtures thereof.

12. The cosmetic, pharmaceutical or dermatological formulation as claimed in claim 1, wherein, the amount of structural units of component a) is from 60.0% to 99.5% by weight, the amount of structural units of component b) is from 0.5% to 20.0% by weight, and the amount of structural units of component c) is from 0% to 20.0% by weight.

13. The cosmetic, pharmaceutical or dermatological formulation as claimed in claim 1, wherein the amount of structural units of component a) is from 80.0% to 98.0% by weight, the amount of structural units of component b) is from 0.7% to 11.0% by weight, and the amount of structural units of component c) is from 0% to 11.0% by weight.

14. The cosmetic, pharmaceutical or dermatological formulation as claimed in claim 3, wherein the weight ratio of structural units originating from the compounds of the formula (Ia) to structural units originating from the compounds of the formula (Ib) in the copolymer is 1.0:0 to 0.8.

15. (canceled)

16. The cosmetic, pharmaceutical or dermatological formulation wherein the copolymer as claimed in claim 1, consists of components a) and b).

17. The cosmetic, pharmaceutical or dermatological formulation wherein the copolymer as claimed in claim 1, consists of components a), b), and c).

18. The cosmetic, pharmaceutical or dermatological formulation as claimed in claim 17, wherein the structural units of component c) are selected from crosslinking structural units.

19. A process for preparing the copolymer as claimed in claim 1, comprising the step of radically polymerizing the monomers.

20. (canceled)

21. A process for thickening oil comprising the step of adding at least one copolymer comprising
a) 60.0%-99.9% by weight of at least one structural unit originating from at least one polymerizable compound selected from group consisting of the structural formulae (Ia) and (Ib) 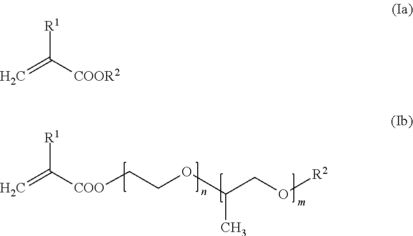
wherein
R¹ is hydrogen or methyl,
R² is a linear or branched saturated alkyl radical, having 12 to 200 carbon atoms, or a linear or branched mono- or polyunsaturated alkenyl radical having 12 to 200 carbon atoms,
and
n and m each independently of one another are an integer from 0 to 200 and the sum of n+m is a number from 1 to 200,
b) 0.1%-20.0% by weight of at least one structural unit, originating from at least one polymerizable quaternary ammonium compound, and
c) 0%-20.0% by weight of at least one nonionic structural unit originating from at least one further polymerizable substance to an oil.

22. A process for conditioning fibers comprising the step of contacting the fibers with at least one cosmetic, pharmaceutical or dermatological formulation as claimed in claim 1.

23. A process for increasing the water resistance of sun protection formulations comprising the step of adding at least one cosmetic, pharmaceutical or dermatological formulation as claimed in claim 1 to the sun protection formulation.

24. A process for increasing the sun protection factor of sun protection formulations comprising the step of adding at least one cosmetic, pharmaceutical or dermatological formulation as claimed in claim 1 to the sun protection formulation.

25. (canceled)
